(19) United States
(12) Patent Application Publication
(10) Pub. No.: US 2006/0036207 A1
(43) Pub. Date: Feb. 16, 2006

(54) System and method for treating glaucoma

(76) Inventors: James P. Koonmen, Santa Clara, CA (US); Norman F. Smith, Albuquerque, NM (US); Jeffry J. Sniegowski, Tijeras, NM (US); Stephen M. Barnes, Albuquerque, NM (US); Paul J. McWhorter, Albuquerque, NM (US); M. Steven Rodgers, Albuquerque, NM (US)

Correspondence Address: MARSH, FISCHMANN & BREYFOGLE LLP, 3151 SOUTH VAUGHN WAY, SUITE 411, AURORA, CO, 80014, US

(21) Appl. No.: 11/209,357

(22) Filed: Aug. 23, 2005

Related U.S. Application Data
(63) Continuation-in-part of application No. 11/095,995, filed on Mar. 31, 2005.
Application No. 11/095,995 is a continuation-in-part of application No. 11/065,183, filed on Feb. 24, 2005.
Application No. 11/065,183 is a continuation-in-part of application No. 10/911,424, filed on Aug. 4, 2004.
(63) Continuation-in-part of application No. 11/023,289, filed on Dec. 24, 2004.
Application No. 11/023,289 is a continuation-in-part of application No. 10/791,396, filed on Mar. 2, 2004.
Application No. 11/023,289 is a continuation-in-part of application No. 10/858,153, filed on Jun. 1, 2004.
(63) Continuation-in-part of application No. 11/048,195, filed on Feb. 1, 2005.
(63) Continuation-in-part of application No. 11/080,075, filed on Mar. 14, 2005.
(63) Continuation-in-part of application No. 11/158,144, filed on Jun. 21, 2005.
(60) Provisional application No. 60/547,252, filed on Feb. 24, 2004.

(51) Int. Cl. A61M 5/00

(52) U.S. Cl. 604/8

(57) ABSTRACT

An integral conduit (407) in the formed of stepped tubing defines at least part of a drainage flow path (408/408′) that accommodates a flow rate of at least about 0.15 microliters/minute/mm²/mm-Hg out of the anterior chamber (284) of the eye (266). One or more flow modules (415) may be disposed within this drainage flow path (408/048′) and are located exteriorly of the eye (266). Each flow module (415) may be in the form of a filter or a pressure regulator. In one embodiment, one flow module (415) in the form of a filter is used in combination with another flow module (415) in the form of a pressure regulator.

### CROSS-REFERENCE TO RELATED APPLICATIONS

This patent application is a continuation-in-part of, and claims priority under 35 U.S.C. §120 to, U.S. patent application Ser. No. 11/095,995, that is entitled “MEMS FILTER MODULE WITH CONCENTRIC FILTERING WALLS,” and that was filed on Mar. 31, 2005. This patent application is also a continuation-in-part of, and claims priority under 35 U.S.C. §120 to, U.S. patent application Ser. No. 11/065,183, that is entitled “GLAUCOMA IMPLANT HAVING MEMS FILTER MODULE,” and that was filed on Feb. 24, 2005, which is a continuation-in-part of, and claims priority under 35 U.S.C. §120 to, U.S. patent application Ser. No. 10/911,424, that is entitled “MEMS FILTER MODULE,” and that was filed on Aug. 4, 2004, and which further claims priority under 35 U.S.C. §119 to U.S. Provisional Patent Application Ser. No. 60/547,252, that is entitled “MEMS FILTER MODULE,” and that was filed on Feb. 24, 2004. This patent application is also a continuation-in-part of U.S. patent application Ser. No. 11/023,289 that is entitled “IMPLANT HAVING MEMS FLOW MODULE WITH MOVABLE, FLOW-CONTROLLING BAFFLE,” and that was filed on Dec. 24, 2004, which is a continuation in part of each of U.S. patent application Ser. No. 10/791,396, that is entitled “MEMS FLOW MODULE WITH FILTRATION AND PRESSURE REGULATION CAPABILITIES,” and that was filed on Mar. 2, 2004, and U.S. patent application Ser. No. 10/858,153, that is entitled “FILTER ASSEMBLY WITH MICROFABRICATED FILTER ELEMENT,” and that was filed on Jun. 1, 2004. This patent application is also a continuation-in-part of, and claims priority under 35 U.S.C. §120, to each of the following applications: U.S. patent application Ser. No. 11/048,195, that is entitled “MEMS FLOW MODULE WITH PIVOTING-TYPE BAFFLE,” and that was filed on Feb. 1, 2005; U.S. patent application Ser. No. 11/080,075, that is entitled “MEMS FLOW MODULE WITH PISTON-TYPE PRESSURE REGULATING STRUCTURE,” and that was filed on Mar. 14, 2005; and U.S. patent application Ser. No. 11/158,144, that is entitled “GLAUCOMA IMPLANT HAVING MEMS FLOW MODULE WITH FLEXING DIAPHRAGM FOR PRESSURE REGULATION,” and that was filed on Jun. 21, 2005. The entire disclosure of each of these related patent applications is incorporated by reference in their entirety herein.

### FIELD OF THE INVENTION

The present invention generally relates to the field of addressing the intraocular pressure of an eye and, more particularly, to providing a drainage flow path out of the anterior chamber of the eye that accommodates a desirably high flow rate through a relatively small space.

### BACKGROUND OF THE INVENTION

High internal pressure within the eye can damage the optic nerve and lead to blindness. There are two primary chambers in the eye—an anterior chamber and a vitreous body that are generally separated by a lens. Aqueous humor exists within the anterior chamber, while vitreous humor exists in the vitreous body. Generally, an increase in the internal pressure within the eye is caused by more fluid being generated within the eye than is being discharged by the eye. The general consensus is that it is excess fluid within the anterior chamber of the eye that is the main contributor to an elevated intraocular pressure.

One proposed solution to addressing high internal pressure within the eye is to install an implant. Implants are typically directed through a wall of the patient's eye so as to fluidly connect the anterior chamber with an exterior location on the eye. There are a number of issues with implants of this type. One is the ability of the implant to respond to changes in the internal pressure within the eye in a manner that reduces the potential for damaging the optic nerve. Another is the ability of the implant to reduce the potential for bacteria and the like passing through the implant and into the interior of the patient's eye, for instance into the anterior chamber.

### BRIEF SUMMARY OF THE INVENTION

A first aspect of the present invention is generally directed to addressing intraocular pressure within an eye. A drainage flow path extends from an anterior chamber of the eye to a first drainage location. A flow of at least about 0.15 microliters/minute/mm²/mm-Hg may progress through this drainage flow path.

Various refinements exist of the features noted in relation to the first aspect of the present invention. Further features may also be incorporated in the first aspect of the present invention as well. These refinements and additional features may exist individually or in any combination. The first aspect is generally directed to providing a desirably high flow rate through a relatively small flow path, which is of course advantageous for the treatment of glaucoma in a number of respects. The maximum cross-sectional area of the drainage flow path defined by its perimeter at any location along the length of the drainage flow path is about 1 mm² in one embodiment, is about 0.5 mm² in another embodiment, and is about 0.2 mm² in yet another embodiment. Although a baseline flow rate of sorts is presented in relation to the first aspect, even higher flow rates are accommodated and in accordance with the following separate embodiments: at least about 0.3 microliters/minute/mm²/mm-Hg; at least about 0.6 microliters/minute/mm²/mm-Hg; at least about 1.2 microliters/minute/mm²/mm-Hg; and about 1.5 microliters/minute/mm²/mm-Hg.

A flow is again directed out of the anterior chamber of the eye to the first drainage location via the drainage flow path. The first drainage location may be any appropriate destination, including without limitation: exteriorly of the eye (e.g., a conjunctival cul-de-sac or the region between an eyelid (upper or lower) and the conjunctiva on the sclera); exteriorly of the cornea of the eye; exteriorly of the sclera of the eye; another location within the eye (e.g., into Schlemm's canal); or into another portion of the body. Preferably, the drainage flow path accommodates flow rates within a range of about 1-5 microliters/minute, while maintaining the pressure within the anterior chamber of the eye within the range of about 5-20 mm-Hg.

The drainage flow path preferably includes what may be characterized as a bacterial retention region. At least certain bacteria and other undesired particulates may be retained within this bacterial retention region before being able to reach the anterior chamber of the eye. This may be viewed as a filtration function. Preferably, all flow between the anterior chamber and the first drainage location must pass through this bacterial retention region. This bacterial retention region may be defined in any appropriate manner. In one embodiment, the bacterial retention region is configured to mechanically obstruct particles of larger than a certain size such that they do not pass through the bacterial retention region and reach the anterior chamber of they eye (e.g., in the form of a filter). For instance, at least a substantial portion of particles larger than about 0.4 microns in one embodiment, larger than about 0.3 microns in another embodiment, larger than about 0.2 microns in another embodiment, and larger than about 0.1 microns in yet another embodiment are retained within the bacterial retention region. Bacteria such as pseudomonas aeruginosa (0.5 micron minimum dimension) and larger bacteria such as staphylococcus aureus (1 micron minimum dimension) should thereby be retained within the bacterial retention region, preferably with a desirably high efficiency rate. However the bacterial retention region may be configured to retain the smallest particle of interest to reduce the potential of the same reaching the anterior chamber of the eye through the drainage flow path. For instance, the bacterial retention region could be configured to retain bacteria such as brevundimonas diminuta (0.35 micron minimum dimension).

The drainage flow path may also include what may be characterized as a flow restriction region. Such a flow restriction region is desirable to reduce the potential of the pressure within the anterior chamber of the eye from decreasing to an undesired level. In one embodiment, the drainage flow path accommodates flow rates out of the anterior chamber within a range of about 1-5 microliters/minute, while maintaining the pressure within the anterior chamber within a range of about 5-20 mm-Hg. This may be viewed as a pressure regulation function. The above-noted bacterial retention region and the flow restriction region can be disposed at the same location within the drainage flow path or may be spaced along the drainage flow path. The bacterial retention region and the flow restriction region may be defined by a common structure (e.g., a pressure regulator that also provides a filtration function, at least when there is no differential pressure across the pressure regulator or when this differential pressure is less than a certain amount; a MEMS device that includes separate filter and pressure regulator sections). The bacterial retention region and flow restriction regions could also be defined by different, spaced structures (e.g., a filter and a separate pressure regulator).

The drainage flow path may be defined by a conduit that is exposed to (e.g., extends into) the anterior chamber of the eye. An anti-bacterial material may be used in the installation of such a conduit. In one embodiment, the conduit is disposed within the anterior chamber, extends through the cornea and then between the sclera and the conjunctiva, and then extends through the conjunctiva into the conjunctival cul-de-sac. Therefore, one end of the conduit may be disposed within the anterior chamber and another end of the conduit may be disposed in a conjunctival cul-de-sac. It may be desirable to promote adhesion and tissue integration between the exterior of the conduit and adjacent scleral and conjunctival tissue. This “adhesion promotion” may be accomplished in any appropriate manner (e.g., through a coating that fosters tissue integration, like a pHEMA hydrogel).

At least one flow module (e.g., a MEMS device) may be disposed within a conduit that defines at least part of the drainage flow path. The term “flow” in relation to this flow module merely means that the flow module accommodates a flow therethrough. Although any such flow module could be disposed directly into such a conduit, one or more housings could also be used to integrate any such flow module with the conduit as well.

A number of general characterizations may be made in relation to the above-noted flow module. Preferably, all flow through the drainage flow path must pass through at least one flow module. Each such flow module may also include at least one hydrophilic surface, such that a large differential pressure is not required to initiate a flow through the flow module (e.g., a differential pressure across the flow module of no more than about 50 mm-Hg (more preferably a differential pressure across the flow module of no more than about 5-10 mm-Hg) should initiate a flow through the module). Preferably, each flow path through the flow module is entirely defined by such a hydrophilic surface. All surfaces of the flow module and any associated housing(s) that are exposed to a fluid when disposed within the conduit may also be configured to reduce the ability of biological materials to attach thereto. In one embodiment, a self-assembled monolayer coating is applied to each such surface.

The flow module may be in the form of a filter. One way to characterize a flow module in the form of a filter is that it includes at least one flow path or multiple flow paths, each being of a fixed size (e.g., a fixed pore size). Another way to characterize a flow module in the form of a filter is that it provides at least a substantially linear increase in a flow therethrough in response to an increase in a differential pressure across the flow module. The filter may be fabricated to filter at least substantially all particles that are larger than about 0.4 microns in one embodiment, that are larger than about 0.3 microns in another embodiment, that are larger than about 0.2 microns in another embodiment, and that are larger than about 0.1 microns in yet another embodiment. The filter may be characterized as having a plurality of filter trap gaps, with the filter being fabricated to attempt to have each of these filter trap gaps be the same size, and with the size of the largest filter trap gap being no more than about 105% of the size of the smallest filter trap gap.

The flow module may also be in the form of a pressure regulator. One way to characterize a flow module in the form of a pressure regulator is that it provides greater than a linear increase in a flow therethrough in response to an increase in a differential pressure across the flow module. Another way to characterize a flow module in the form of a pressure regulator is that it includes at least one element that moves in response to a change in the differential pressure across the flow module. This pressure regulator may be configured to provide a filtering function in accordance with the foregoing when there is no differential pressure across the flow module or when the differential pressure across the flow module is less than a certain amount. It may also be possible to fabricate a single MEMS device having a filter section and a pressure regulator section disposed in series and in any order (e.g., a filter section defined by two or more structural layers and a pressure regulator section defined by two or more structural layers).

More than one flow module could be disposed within a conduit that defines at least part of the drainage flow path. One of these flow modules could be in the form of a filter as described above, while another of these flow modules could be in the form of a pressure regulator as described above. In one embodiment, multiple flow modules are disposed in series (i.e., a flow passes sequentially through such flow modules) when positioned within the conduit.

Any flow module disposed within a conduit that defines at least part of the drainage flow path may be replaceable, and preferably this may be undertaken without the need to withdraw the conduit from the anterior chamber (e.g., no need for a surgical procedure to replace the flow module). A number of actions may be undertaken in preparation for the replacement of a flow module. It may be desirable to apply an anti-bacterial material to the eye and any other relevant surfaces in preparation for the replacement. The conduit also may be at least substantially occluded to facilitate the replacement of the flow module. The conduit could be at least substantially occluded at one location “upstream” of the flow module (in the direction of the anterior chamber) being replaced before attempting to withdraw the flow module, an open end of the conduit could be at least substantially occluded after the flow module has been withdrawn from the conduit, or both. What of course is desired is for the drainage flow path to be externally restricted or blocked during replacement of the flow module to reduce the potential for hypotony (e.g., experiencing an intraocular pressure of less than about 5 mm-Hg) and also to reduce the potential of bacteria being able to progress through the drainage flow path and reach the anterior chamber of the eye.

There are a number of options that could be used to provide for a replacement of the flow module. A portion of the conduit could be expanded in any appropriate manner to allow for the removal of a flow module though an open end of the conduit. For instance, a device could be directed into an open end of the conduit, and thereafter could be expanded to in turn expand the conduit from “about” the flow module. The flow module could then be withdrawn from the conduit in any appropriate manner (e.g., mechanically, by a vacuum). Another option would be to replace the flow module by replacing an entire section of the conduit that is disposed exteriorly of the eye. In either case, the flow module may be replaced without removing the conduit from the anterior chamber of the eye (e.g., a surgical procedure should not be required to replace a flow module).

Although the conduit may be of any appropriate configuration (e.g., having a constant outer and inner diameter), in one embodiment the conduit includes first and second conduit sections, where the first conduit section is directed through the eye and is exposed to (e.g., extends into) the anterior chamber, where the entirety of the second conduit section is disposed exteriorly of the eye, and where the drainage flow path within the first conduit section has a smaller cross-sectional profile than the drainage flow path within the second conduit section. The “cross-sectional profile” is taken perpendicularly to the length dimension of the relevant conduit section. The cross-sectional profile would be the cross-sectional area encompassed by the perimeter of the inner surface of the conduit that defines at least part of the drainage flow path. In the case where the first and second conduit sections are each cylindrical structures, the first conduit section would have a smaller inner diameter than the second conduit section to provide the noted smaller cross-sectional profile. In any case, preferably the conduit is an integral structure (e.g., a stepped diameter or tapered tube), with no joint of any kind between the first and second conduit sections. That is, preferably the conduit having the noted first and second conduit sections is of one-piece construction.

A number of characterizations may be made in relation to the above-noted conduit having first and second conduit sections. One is that the portion of the first conduit section that extends from the anterior chamber thereafter progresses between the sclera and the conjunctiva, and then extends through the conjunctiva “under” an eyelid. The entire second conduit section thereby may be disposed in the above-noted conjunctival cul-de-sac. One or more flow modules of the above-noted type may be disposed within the second conduit section. The second conduit section may also be used to establish an interconnection with a third conduit section that houses at least one flow module in accordance with the foregoing. An appropriate coupling may establish the interconnection between the second and third conduit sections. Although this coupling could utilize a smooth exterior surface (e.g., cylindrical), preferably the coupling includes at least two protuberances (e.g., barbs) on its exterior surface to enhance the mechanical purchase of the coupling with each of second and third conduit sections. Replacement of a flow module could then entail simply disconnecting the third conduit section from the second conduit section (e.g., by disconnecting the coupling from the second conduit section, by disconnecting the third conduit section from the coupling, or both). The third conduit section with the flow module(s) remaining therein could then be properly disposed of as a single unit. Alternatively, the flow module(s) within the now disconnected third conduit section could simply be replaced within the same third conduit section. In any case, a third conduit section (the original or a new one) with a new flow module(s) therein may thereafter be re-joined with the second conduit section via the coupling.

A second aspect of the present invention is generally directed to addressing intraocular pressure within an eye. A conduit defines a drainage flow path, and is configured so as to extend from the anterior chamber of the eye to a suitable drainage location. First and second flow modules are disposed within this conduit. The various features discussed above in relation to the first aspect may be used by this second aspect, individually and in any combination.

A third aspect of the present invention is generally directed to addressing intraocular pressure within an eye. A conduit defines a drainage flow path, and is configured so as to extend from the anterior chamber of the eye to a suitable drainage location. This conduit includes first and second conduit sections, where the first conduit section is directed through the eye and is exposed to (e.g., directed into) the anterior chamber, where the entirety of the second conduit section is disposed exteriorly of the eye, and where the drainage flow path within the first conduit section has a smaller cross-sectional profile than the drainage flow path within the second conduit section. The “cross-sectional profile” is taken perpendicularly to the length dimension of the relevant conduit section. In the case where the first and second conduit sections are each cylindrical structures, the first conduit section would have a small inner diameter than the second conduit section to provide the noted smaller cross-sectional profile. The various features discussed above in relation to the first aspect may be used by this third aspect, individually and in any combination.

A fourth aspect of the present invention is generally directed to addressing intraocular pressure within an eye. A conduit defines a drainage flow path, and is configured so as to extend from the anterior chamber of the eye to a suitable drainage location. A MEMS device is disposed within this conduit and includes at least one, but more preferably a plurality of gaps through which a flow may be directed. Each of these gaps is intended to be of the same size. Any variation in the size of these gaps is such that the size of largest gap is no more than about 105% of the size of the smallest gap.

Various refinements exist of the features noted in relation to the fourth aspect of the present invention. Further features may also be incorporated in the fourth aspect of the present invention as well. These refinements and additional features may exist individually or in any combination. The various features discussed above in relation to the first aspect may be used by the fourth aspect, individually and in any combination. The size of the gaps may be of a fixed dimension in the case of the fourth aspect. The size of these gaps may also be adjustable. For instance, the size of the gaps may increase when the MEMS device is exposed to a differential pressure to accommodate an increased flow therethrough. The size variation of the gaps noted above would thereby be applicable when there is no differential pressure across the MEMS device. The MEMS device could include a plurality of groups of gaps in accordance with the fourth aspect. Each gap within a given group would be of the same size, subject to the above-noted size variation. The size of the gaps could be different from group-to-group. That is, one group of gaps could be of one specified size, and another group of gaps could be of another specified size.

A fifth aspect of the present invention is directed to withdrawing a flow module out from within a conduit. This conduit defines at least part of a drainage flow path for a biological fluid (e.g., to accommodate the flow of aqueous humor out of the anterior chamber of the eye, which is then directed to an appropriate drainage location). An extraction device is directed into an open end of the conduit, and is thereafter expanded to release the flow module from the interior of the conduit (e.g., to reduce the force required to move the flow module relative to the conduit). The flow module may be withdrawn out of the conduit as the extraction device is withdrawn out of the conduit.

Various refinements exist of the features noted in relation to the fifth aspect of the present invention. Further features may also be incorporated in the fifth aspect of the present invention as well. These refinements and additional features may exist individually or in any combination. The various features discussed above in relation to the first aspect may be used by this fifth aspect, individually and in any combination. The extraction device may be in the form of a first assembly having first and second members. The second member includes a head that is larger than a cross-sectional profile of at least a portion of a passageway that extends into the first member. The first assembly may be directed into the conduit, and thereafter the head of the second member may be moved relative to the passageway of the first member. This expands the first member, which in turn expands the conduit a sufficient amount to allow for removal of the flow module. In one embodiment, the first and second members are each in the form of hollow tubes, and the first member includes a plurality of slots that extend from one of its ends and that define a plurality of segments that may be expanded when engaged by the head of the second member as the second member is advanced relative to the passageway of the first member.

There are a number of ways in which the flow module may be actually withdrawn out of the conduit once the conduit has been expanded in accordance with the foregoing. A suction force could be applied via the first assembly to capture the flow module within the expanded first member, to retain the flow module against the first assembly, or to exert a sufficient force on the flow module to move the same relative to the conduit as the first assembly is withdrawn out of the conduit (leaving the first member in its expanded state or otherwise). Another option would be to direct a second assembly into the first assembly. In one embodiment, the second assembly is of the same general configuration as the first assembly. In any case, this second assembly may have third and fourth members. The fourth member includes a head that is larger than a cross-sectional profile of at least a portion of a passageway that extends into the third member. The second assembly may be directed beyond an end of the first assembly and into an open end of a housing associated with the flow module. Thereafter, the head of the fourth member may be advanced relative to the passageway of the third member. This expands the third member into engagement with the interior of the noted flow module housing, and which should provide a sufficient mechanical engagement force to allow the first and second assemblies to be withdrawn out of the conduit to remove the flow module from within the conduit.

A sixth aspect of the present invention is directed to addressing intraocular pressure within an eye. A conduit extends from the anterior chamber to an appropriate drainage location. A flow module is disposed within this conduit at a location exteriorly of the eye. At least a portion of the conduit between the anterior chamber of the eye and the flow module is at least substantially occluded. Thereafter the flow module is replaced.

Various refinements exist of the features noted in relation to the sixth aspect of the present invention. Further features may also be incorporated in the sixth aspect of the present invention as well. These refinements and additional features may exist individually or in any combination. Initially, the various features discussed above in relation to the first aspect may be used by this sixth aspect, individually and in any combination.

The conduit may be of any appropriate size, shape, and/or configuration. For instance, the conduit could be an integral or one-piece structure, the conduit could be defined by a plurality of separate conduit sections that are appropriately interconnected, the conduit could include a flow path of a uniform size therethrough, or the conduit could include a flow path having at least two different sections of a different size.

The flow module could be of any appropriate size, shape, and/or configuration, and may provide any desired/required function or combination of functions. The features of the flow module(s) discussed above in relation to the first aspect are applicable to this sixth aspect, and may be used individually or in any combination. The fifth aspect may be used in relation to the replacement of the flow module in accordance with this sixth aspect as well. The flow module could also be replaced in the manner discussed above in relation to the first aspect as well. Preferably, the flow module is replaced without removing the conduit from the anterior chamber of the eye.

The conduit may be at least substantially occluded at one or more locations between the anterior chamber and the flow module being replaced. Any appropriate way of occluding the conduit may be utilized.

### BRIEF DESCRIPTION OF THE SEVERAL VIEWS OF THE DRAWING

FIG. 1 is a cross-sectional view of a schematic of a glaucoma drainage device installed in an eye.

FIG. 2 is a cross-sectional view of one embodiment of the glaucoma drainage device of FIG. 1.

FIG. 3A is a perspective view of an extraction tool for removing a flow module from a conduit.

FIG. 3B is a perspective view of the extraction tool of FIG. 3A when being used to expand a conduit for the removal of the flow module.

FIG. 4 is a cross-sectional view of another embodiment of the glaucoma drainage device of FIG. 1.

FIG. 4A is a side view of the coupling used by the glaucoma drainage device of FIG. 4.

FIGS. 4B-E are side views of alternative designs for couplings that may be used by the glaucoma drainage device of FIG. 4.

FIG. 5 is a cross-sectional view of another embodiment of the glaucoma drainage device of FIG. 1.

FIG. 6 is a schematic of another embodiment of a glaucoma drainage device.

FIG. 7 is a cross-sectional view of another embodiment of a glaucoma drainage device.

FIG. 8A is an exploded, perspective view of one embodiment of a flow assembly that uses a flow module.

FIG. 8B is a perspective view of the flow assembly of FIG. 8A in an assembled condition.

FIG. 9A is an exploded, perspective of another embodiment of a flow assembly that uses a flow module.

FIG. 9B is a perspective view of the flow assembly of FIG. 9A in an assembled condition.

FIG. 10A is an exploded, perspective of another embodiment of a flow assembly that uses a flow module.

FIG. 10B is a perspective view of the flow assembly of FIG. 10A in an assembled condition.

FIG. 11 is a side view of a plurality of layers that may be used by one embodiment of a surface micromachining fabrication technique.

FIG. 12 is a perspective view of one embodiment of a MEMS flow module that utilizes a plurality of concentrically disposed, annular filter walls to provide at least a filtering function.

FIG. 13 is a cross-sectional, exploded, perspective view of the MEMS flow module of FIG. 12, taken along a plane between its first and second plates so as to not intersect with the various annular filter walls that extend from the second plate toward, but not to, the first plate, and with the second plate having been pivoted away from the first plate.

FIG. 14 is a cross-sectional view that illustrates the spacing between the first plate and the annular filtering walls of the second plate in the case of the MEMS flow module of FIG. 12.

FIG. 15 is a cross-sectional, exploded, perspective view of the MEMS flow module of FIG. 12, taken along a plane that extends between its second plate and an annular support or ring that sandwiches the second plate between this annular support and the first plate, with this annular support having been pivoted away from the second plate.

FIG. 16A is a perspective view of one embodiment of a MEMS flow module that provides at least a pressure-regulation function.

FIG. 16B is a cross-sectional, exploded, perspective view of the MEMS flow module of FIG. 16A.

FIG. 16C is a cross-sectional view through one of the baffles of the MEMS flow module of FIG. 16A.

### DETAILED DESCRIPTION OF THE INVENTION

The present invention will now be described in relation to the accompanying drawings that at least assist in illustrating its various pertinent features. Generally, the present invention relates to addressing the intraocular pressure of an eye. More specifically, what may be characterized as an implant, shunt, or drainage device is directed through the eye for exposure to the anterior chamber of the eye, and extends from the anterior chamber to an appropriate drainage location or destination to accommodate a desired flow of aqueous humor out of the anterior chamber. The term “implant,” as used herein, means a device that is at least partially disposed within an appropriate biological mass. The entire implant could be disposed within a biological mass. Another option would be for part of the implant to be disposed within a biological mass, and for another part of the implant to be disposed externally of the biological mass or at least provide for fluid communication externally of the biological mass (e.g., by interfacing with the environment).

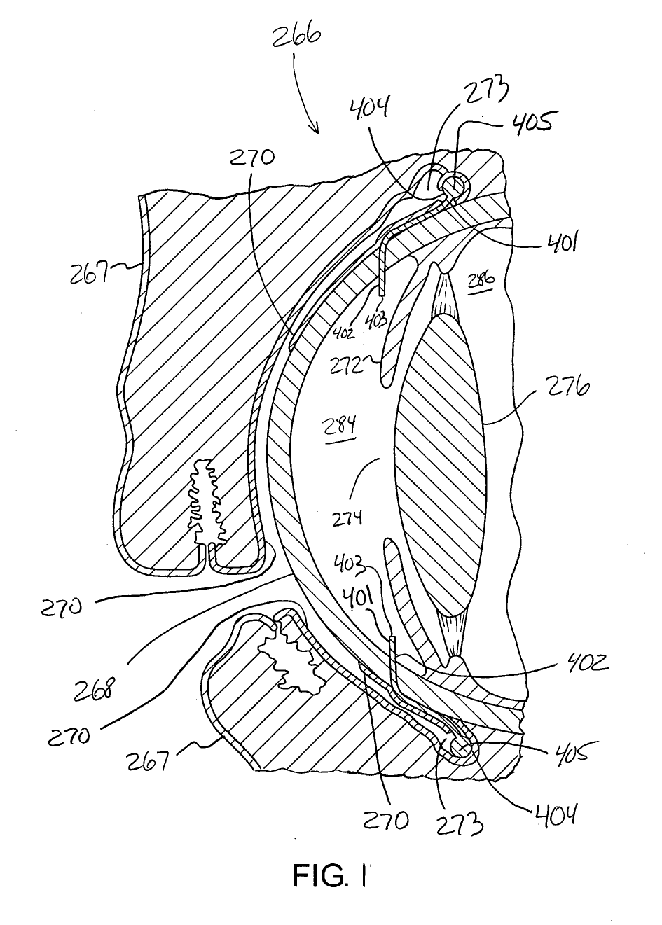

Various portions of an eye 266 are identified in FIG. 1, including the cornea 268, conjunctiva or conjunctival layer 270, iris 272, pupil 274, lens 276, anterior chamber 284, vitreous body 286, and eyelids 267. The portion of the conjunctiva 270 that is disposed over the sclera and that extends along the inner surface of the eyelids 267 defines what may be characterized as a conjunctival cul-de-sac 273. A glaucoma drainage device, implant, or shunt 401 (“GDD 401”) is also schematically illustrated in FIG. 1 in two representative installation positions. Although a pair of GDDs 401 could be installed within the same eye 266 as shown in FIG. 1, typically only a single GDD 401 would be used for treating a given eye 266.

There are a number of particularly desirable characteristics of the GDD 401. Generally, the GDD 401 includes an internal drainage flow path that accommodates a desirably high flow rate and is of a relatively small cross-sectional profile (perpendicularly to its length). The maximum cross-sectional area of a drainage flow path through the GDD 401 and defined by the perimeter of its inner surface is about 1 mm² in one embodiment, is about 0.5 mm² in another embodiment, and is about 0.2 mm² in yet another embodiment. Preferably, the GDD 401 accommodates flow rates out of the anterior chamber 284 of the eye 266 within a range of about 1-5 microliters/minute, while maintaining a pressure within the anterior chamber 284 within a range of about 5-20 mm-Hg. The GDD 401 also accommodates a flow out of the anterior chamber 284 of at least about 0.15 microliters/minute/mm²/mm-Hg. Even higher flow rates may be accommodated through the GDD 401 and in accordance with the following separate embodiments: at least about 0.3 microliters/minute/mm²/mm-Hg; at least about 0.6 microliters/minute/mm²/mm-Hg; at least about 1.2 microliters/minute/mm²/mm-Hg; and about 1.5 microliters/minute/mm²/mm-Hg.

The drainage flow path through the GDD 401 preferably includes what may be characterized as a bacterial retention region. At least certain bacteria and other undesired particulates may be retained within this bacterial retention region before being able to reach the anterior chamber 284 of the eye 266. This may be viewed as a filtration function. Preferably, all flow between the anterior chamber 284 and the desired drainage location must pass through this bacterial retention region. This bacterial retention region may be defined in any appropriate manner. In one embodiment, the bacterial retention region is configured to mechanically obstruct particles of larger than a certain size such that they do not pass through the bacterial retention region and reach the anterior chamber 284 of the eye 266 (e.g., in the form of a filter). For instance, at least a substantial portion of particles larger than about 0.4 microns in one embodiment, larger than about 0.3 microns in another embodiment, larger than about 0.2 microns in another embodiment, and larger than about 0.1 microns in yet another embodiment are retained within the bacterial retention region of the GDD 401. Bacteria such as pseudomonas aeruginosa (0.5 micron minimum dimension) and larger bacteria such as staphylococcus aureus (1 micron minimum dimension) should thereby be retained within the bacterial retention region of the GDD 401, preferably with a desirably high efficiency rate. However, the bacterial retention region may be configured to retain the smallest particle of interest to reduce the potential of the same from reaching the anterior chamber 284 of the eye 266. For instance, the bacterial retention region could be configured to retain brevundimonas diminuta (0.35 micron minimum dimension).

The drainage flow path through the GDD 401 may also include what may be characterized as a flow restriction region. Such a flow restriction region is desirable to reduce the potential of the pressure within the anterior chamber 284 of the eye 266 from decreasing to an undesired level. In one embodiment, the drainage flow path accommodates flow rates out of the anterior chamber 284 within a range of about 1-5 microliter/minute, while maintaining the pressure within the anterior chamber 284 within a range of about 5-20 mm-Hg as noted. This may be viewed as a pressure regulation function. The above-noted bacterial retention region and the flow restriction region can be disposed at the same location within the drainage flow path or may be spaced along the drainage flow path. The bacterial retention region and the flow restriction region may be defined by a common structure (e.g., a pressure regulator that also provides a filtration function, at least when there is no differential pressure across the pressure regulator or when this differential pressure is less than a certain amount; a MEMS device that includes separate filter and pressure regulator sections). The bacterial retention region and flow restriction regions could also be defined by different, spaced structures (e.g., a filter and a separate pressure regulator).

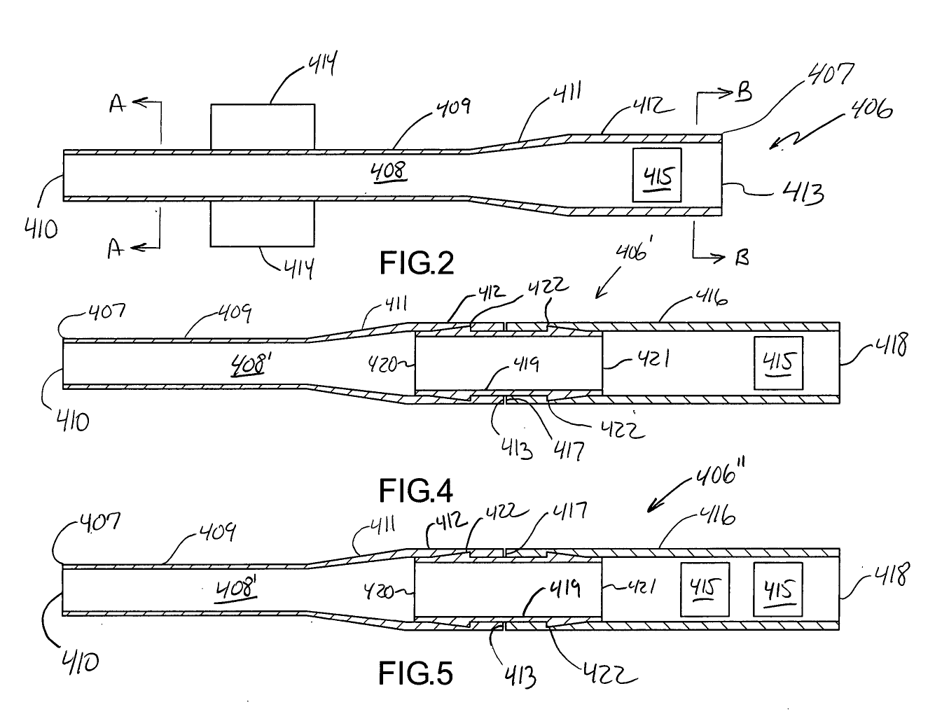

The GDD 401 includes an internal drainage flow path (not shown) that extends through both a first section 402 and a second section 404 of the GDD 401. The first section 402 includes a first open end 403 that is disposed within the anterior chamber 284 of the eye 266. The first section 402 extends from this first open end 403 through the cornea 268. Thereafter, the first section 402 is directed between the cornea 268 and the conjunctiva 270 until it passes through the conjunctiva 270 and into a conjunctival cul-de-sac 273. The entire second section 404 of the GDD 401 is disposed within this conjunctival cul-de-sac 273, which enhances the cosmetic appearance of the patient. As will be discussed in more detail below in relation to the embodiments of FIGS. 2, 4, and 5, the first section 402 has a smaller outer diameter than the second section 404. This reduces irritation of the eye 266, enhances the comfort of the patient, and makes the GDD 401 more minimally perceivable by others.

One embodiment of a glaucoma drainage device, implant or shunt (“GDD”) is illustrated in FIG. 2 and is identified by reference numeral 406. The GDD 406 of FIG. 2 is one specific design for the GDD 401 of FIG. 1. The GDD 406 is generally in the form of a conduit 407 that is sufficiently pliable or flexible to be installed in the manner described herein, and which may be formed from any appropriate material (e.g., silicone, polyurethane). It may be desirable for at least part of the conduit 407 to at least somewhat adhere to an interfacing portion of the eye 266 when installed (e.g., where the conduit 407 penetrates the conjunctiva 270 within the relevant conjunctival cul-de-sac 273). Any way of promoting adhesion of the eye 266 to the conduit 407 at the desired location(s) may be utilized, such as by applying any appropriate coating or the like to an exterior surface of the conduit 407.

The conduit 407 has an internal drainage flow path 408 that extends between a first open end 410 and a second open end 413. The drainage flow path 408 extends through a first conduit section 409 (corresponding with the first section 402 of the GDD 401 of FIG. 1), a transition section 411, and a second conduit section 412 (corresponding with the second section 404 of the GDD 401 of FIG. 1). The free end of the first conduit section 409 may be configured in any appropriate manner (e.g., to facilitate installation of the GDD 406). The second conduit section 412 is oriented transversely when the GDD 406 is installed (e.g., one could be looking into the second open end 413 in the view presented in FIG. 1). A suture crossbar 414 may be disposed over and appropriately anchored to (e.g., via adhesive) the first conduit section 409, and may be sutured to the eye 266 in any appropriate manner. The suture crossbar 414 may be of any appropriate size, shape, and/or configuration, and further may be formed from any appropriate material (e.g., silicone).

A flow module 415 is disposed within the second conduit section 412 and assumes an at least generally fixed position. Since the entirety of the second conduit section 412 is located exteriorly of the eye 266 when the GDD 406 is installed, so too is the flow module 415. This may offer one or more advantages, including without limitation accommodating replacement of the flow module 415 in a manner that will be discussed in more detail below. That is, the flow module 415 may be accessed through the second open end 413 of the GDD 406 while the second conduit section 412 is within a conjunctival cul-de-sac 273. The flow module 415 could be directly disposed within the conduit 407 as shown, or one or more housings could be used to integrate the flow module 415 with the conduit 407 (the flow module 415 and any integrating housing collectively defining a flow assembly).

The flow module 415 is only schematically illustrated in FIG. 2, and may be that which allows the GDD 406 to realize one or more of the characteristics/parameters discussed above in relation to the GDD 401 of FIG. 1. Generally, the flow module 415 accommodates a flow therethrough when positioned within the drainage flow path 408. Preferably all flow through the drainage flow path 408 must pass through the flow module 415. The flow module 415 may provide any desired/required function in relation to this flow, and may be of any appropriate size, shape, and/or configuration. For instance, the flow module 415 may be in the form of a filter. One characterization in this regard is that the flow module 415 includes at least one, and more preferably a plurality of flow paths, each being of a fixed size (e.g., having a fixed pore size). Another characterization in this regard is that the flow module 415 provides at least a substantially linear increase in a flow therethrough in response to the development of or a change in the differential pressure across the flow module 415 (e.g., from an increase in the pressure within the anterior chamber 284). The flow module 415 may be fabricated to filter particles that are larger than about 0.4 microns in one embodiment, that are larger than about 0.3 microns in another embodiment, that are larger than about 0.2 microns in another embodiment, and that are larger than about 0.1 microns in yet another embodiment. However, the flow module 415 may be configured to retain the smallest particle of interest to reduce the potential of the same from reaching the anterior chamber 284 of the eye 266. The flow module 415 may be characterized as having a plurality of filter trap gaps, with the flow module 415 being fabricated to attempt to have each of these filter trap gaps be the same size, and with the size of the largest gap being no more than about 105% of the size of the smallest gap.

The flow module 415 may be configured to retain at least a substantial portion of specific organisms. For instance, the flow module 415 could be configured to filter pseudomonas aeruginosa (0.5 micron minimum dimension), staphylococcus aureus (1 micron minimum dimension), and the like (e.g., to reduce the potential of such organisms reaching the anterior chamber of the eye through the GDD 406 by being retained within the flow module 415). Smaller organisms could be targeted for filtration by the flow module 415 as well, for instance brevundimonas diminuta (0.35 micron minimum dimension), such that at least a substantial portion of these organisms are retained within the filter module 415.

The flow module 415 may also be in the form of a pressure regulator. One characterization in this regard is that the flow module 415 provides greater than a linear increase in a flow therethrough in response to the development of or a change in the differential pressure across the flow module 415 (e.g., from an increase in the pressure within the anterior chamber 284). Another characterization in this regard is that the flow module 415 includes at least one element that moves in response to the development of or a change in the differential pressure across the flow module 415 to accommodate an increased flow through the flow module 415. The flow module 415 may be configured to provide a filtering function in accordance with the above-noted particle sizes when there is no differential pressure across the flow module 415 or when the differential pressure across the flow module 415 is less than a certain amount (e.g., a filtering function in accordance with the foregoing may be available for differential pressures of no more than about 15 mm-Hg). Yet another option would be for the flow module 415 to have filter and pressure regulating sections disposed in series and in any order.

The flow module 415 may incorporate a number of other desirable features. One is that the flow module 415 may be self-wetting, in that a large differential pressure is not required to initiate a flow through the flow module 415. In one embodiment, a differential pressure across the flow module 415 of no more than about 50 mm-Hg (more preferably a differential pressure across the flow module 415 of no more than about 5-10 mm-Hg) should initiate a flow through the flow module 415. One way in which this may be accomplished is by having all surfaces of each flow path through the flow module 415 be hydrophilic. Another desirable feature that may be utilized by the flow module 415 is that the potential for bio-fouling (e.g., the attachment of biological cells thereto) may be reduced in any appropriate manner. In one embodiment, all surfaces of the flow module 415 that are exposed to a flow through the drainage flow path 408 may be configured to reduce the ability of biological materials to attach to the flow module 415. In one embodiment, a self-assembled monolayer coating is applied to all surfaces of the flow module 415 that may be exposed to a flow through the drainage flow path 408.

There are also a number of features of note in relation to the conduit 407 for the GDD 406. Preferably, the conduit 407 is an integral structure or of one-piece construction. That is, preferably there is no joint between the first conduit section 409 and the transition section 411, nor is there a joint between the transition section 411 and the second conduit section 412. Moreover, the conduit 407 is sized/configured for its use as a glaucoma drainage device 406. Generally, the perimeter of the first conduit section 409 is smaller than the perimeter of the second conduit section 412. In the case where the first conduit section 409 and the second conduit section 412 are in their preferred form of cylindrical structures (having an inner cylindrical surface and an outer cylindrical surface), the outer diameter of the first conduit section 409 would be smaller than the outer diameter of the second conduit section 412. Another way to characterize the size of the first conduit section 409 and second conduit section 412 is in relation to their respective cross-sectional profiles, taken perpendicularly to their respective length dimensions. The cross-sectional profile of the first conduit section 409 (e.g., taken along line A-A in FIG. 2) is smaller than the cross-sectional profile of the second conduit section 412 (e.g., taken along line B-B in FIG. 2). In the illustrated embodiment, the cross-sectional profile of the first conduit section 409 is constant along its entire length, and the cross-sectional profile of the second conduit section 412 is constant along its entire length, although such need not be the case. The maximum cross-sectional area of the drainage flow path 408 through the second conduit section 412 and defined by its perimeter is about 1 mm² in one embodiment, is about 0.5 mm² in another embodiment, and is about 0.2 mm² in yet another embodiment.

The dimensions of the first conduit section 409 and the second conduit section 412 may be of any appropriate value and may be adapted for the eye 266 in which the GDD 406 is installed. In one embodiment: the outer diameter of the first conduit section 409 is about 0.64 mm; the inner diameter of the first conduit section 409 is about 0.3 mm; the length of the first conduit section 409 is established by the surgeon; the length of the transition section 411 is preferably less than about 2 mm; the outer diameter of the second conduit section 412 is about 0.9 mm; the inner diameter of the second conduit section 412 is about 0.46 mm; and the length of the second conduit section 412 is about 1 mm.

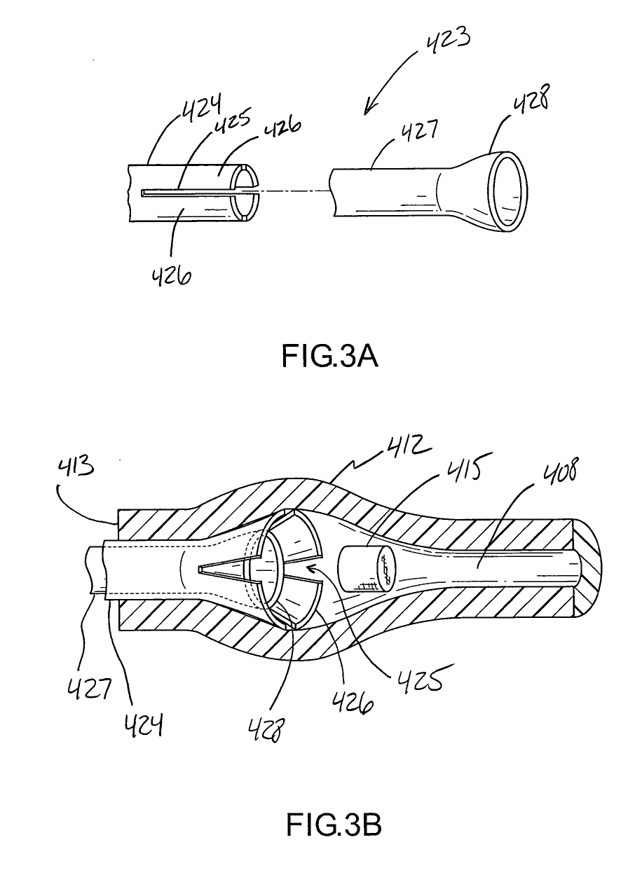

The flow module 415 may be replaced by expanding a relevant portion of the second conduit section 412, and thereafter withdrawing the flow module 415 through the second open end 413 in any appropriate manner (e.g., mechanically, using suction). An extraction tool 423 is illustrated in FIGS. 3A-B, and may be used to expand the second conduit section 412 to facilitate removal of the flow module 415. The extraction tool 423 includes an outer tube 424 having a plurality of slots 425 that extend along a portion of its length and that define a plurality of segments 426. An inner tube 427 having a head or flared end 428 is disposable within the outer tube 424. The outer diameter of the head 428 of the inner tube 427 is larger than the inner diameter of at least a portion of the outer tube 424, but may be the same as or slightly less than the inner diameter of the second conduit section 412. The extraction tool 423 is disposed through the second open end 413 of the conduit 407. The head 428 of the inner tube 427 could extend beyond the end of the outer tube 424 if the outer tube 424 had a constant inner diameter. If a relevant portion of the inner diameter of the outer tube 424 was tapered or stepped, or so as to otherwise reduce the cross-sectional profile of a relevant portion of the interior of the outer tube 424, the head 428 could actually start out within the outer tube 424. In any case, preferably, the end of the extraction tool 423 is disposed at least in the vicinity of the flow module 415 within the second conduit section 412. Thereafter, the head 428 of the inner tube 427 is advanced relative to the outer tube 424 by axially advancing the inner tube 427 relative to the outer tube 424 in any appropriate manner. This expands the segments 426 of the outer tube 424 (by the head 428 engaging a “smaller inner diameter” portion of the outer tube 424), which in turn expands the corresponding portion of the second conduit section 412, all as illustrated in FIG. 3B. This should provide an adequate space to allow the flow module 415 to be moved axially relative to the second conduit section 412 of the conduit 407.

A number of options may be utilized to actually move the flow module 415 out of the second conduit section 412 once released therefrom. A suction force could be applied through the inner tube 427 of the extraction tool 423 to engage the flow module 415 against the extraction tool 423 (e.g., within the outer tube 424; within the inner tube 427), but to in any case move the flow module 415 axially relative to the second conduit section 412 in the direction of the second open end 413. The outer tube 424 could be withdrawn from the second conduit section 412 while remaining in its expanded condition by maintaining the relative position of the inner tube 427 relative to the outer tube 424, although such may not be required in all instances. In any case, suction could continue to be applied during the withdrawal of the extraction tool 423 to retract the flow module 415 out from the second conduit section 412.

Another option would be to mechanically engage the flow module 415 after the extraction tool 423 has expanded the second conduit section 412 (FIG. 3B) to release the flow module 415 from the second conduit section 412. Consider the case where the flow module 415 is disposed within a housing, that an open end of the housing faces the extraction tool 423, and that the flow module 415 is spaced back from this particular open end. With a first extraction tool 423 having already expanded the second conduit section 412 in the noted manner (FIG. 3B), a second extraction tool 423 could be directed through the first extraction tool 423 and into the open end of the noted housing. The inner tube 427 of the second extraction tool 423 could be moved relative to the outer tube 424 of the second extraction tool 423 to expand the same and into engagement with an interior surface of the noted housing. This then provides a mechanical engagement of the second extraction tool 423 with the housing in which the flow module 415 is disposed. The first and second extraction tools 423 could then be simultaneously withdrawn from the second conduit section 412 (preferably with the outer tube 424 of the first extraction tool 423 continuing to expand the second conduit section 412 to at least some degree, although such may not be required). The described procedure could be reversed to install a flow module 415 within the second conduit section 412 of the conduit 407.

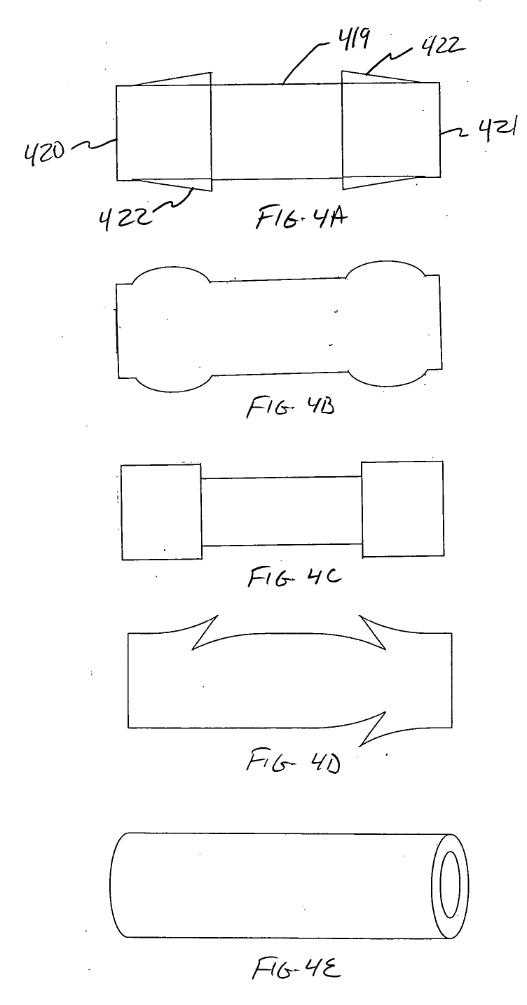

Another embodiment of a glaucoma drainage device, implant, or shunt (“GDD”) is illustrated in FIG. 4 and is identified by reference numeral 406′. The GDD 406′ of FIG. 4 is another specific design for the GDD 401 of FIG. 1. Corresponding components between the embodiments of FIGS. 2 and 4 are identified by the same reference numeral. The suture crossbar 414 is not shown for the GDD 406′, but could be implemented in the above-noted manner. The primary difference between the GDD 406′ of FIG. 4 and the GDD 406 of FIG. 2 is that a third conduit section 416 is interconnected with the second conduit section 412 of the conduit 407, and this third conduit section 416 houses the flow module 415. Once again, the flow module 415 could be directly disposed in the third conduit section 416, or one or more housings could be used to integrate these two structures. The drainage flow path 408′ thereby extends not only through the conduit 407, but also through the third conduit section 416 (between a first open end 417 and a second open end 418 of the third conduit section 416).

A coupling 419 is used to interconnect the second conduit section 412 with the third conduit section 416. Part of the drainage flow path 408′ thereby extends between the first open end 420 and the second open end 421 of the coupling 419. The first open end 420 of the coupling 419 is directed into the second conduit section 412 through its second open end 413. One or more protuberances 422 on the exterior of the coupling 419 may provide at least somewhat of a press or interference fit (a mechanical purchase) between the second conduit section 412 and the coupling 419. The second open end 421 of the coupling 419 is also directed into the third conduit section 416 through its first open end 417. One or more protuberances 422 on the exterior of the coupling 419 may also provide at least somewhat of a press or interference fit (a mechanical purchase) between the third conduit section 16 and the coupling 419.

The cross-sectional profile of the drainage flow path 408′ within the third conduit section 416 may be the same as the cross-sectional profile of the drainage flow path 408′ within the second conduit section 412, although such is not required. The third conduit section 416 may be of any appropriate length, may be of any appropriate size, shape, and/or configuration, and may be formed from any appropriate material (e.g., the same materials noted above in relation to the conduit 407). In the illustrated embodiment, the third conduit section 416 is cylindrical, has about the same inner and outer diameters as the second conduit section 412, and has a length of about 5 mm.

The coupling 419 may be of any appropriate size, shape, and/or configuration, and may be formed from any appropriate material (e.g., stainless steel). The coupling 419 should be long enough so as to be sufficiently engaged with each of the second conduit section 412 and the third conduit section 416 (e.g., a length of about 2 mm in one embodiment). In the illustrated embodiment, the coupling 419 is a cylindrical structure. The protuberances 422 may be of any appropriate size, shape, and/or configuration as well. FIG. 4A is an enlarged view of the coupling 419. FIGS. 4B-4D are other embodiments of couplings that may be used in place of the coupling 419 and which use various other protuberance configurations. It may be such that the exterior surface of the coupling 419 will not need any protuberances at all for interfacing with the second conduit section 412 and the third conduit section 416. This variation is illustrated in FIG. 4E.

The flow module 415 is disposed within the third conduit section 416 between its first open end 417 and its second open end 418, and thereby is disposed within the drainage flow path 408′. This may facilitate replacement of the flow module 415. One option would be to disconnect the third conduit section 416 from the coupling 419. Another option would be to disconnect the coupling 419 from the second conduit section 412. In any case, the third conduit section 416 with the flow module 415 contained therein could then be removed from the conjunctival cul-de-sac 273. This third conduit section 416 could simply be discarded and replaced by a new third conduit section 416 with a flow module 415 contained therein (i.e., a section of the actual drainage flow path 408′ is replaced in this instance). Alternatively, the flow module 415 alone could be replaced, using the same third conduit section 416. The described procedure could be reversed to install a third conduit section 416 with a replacement flow module 415 therein.

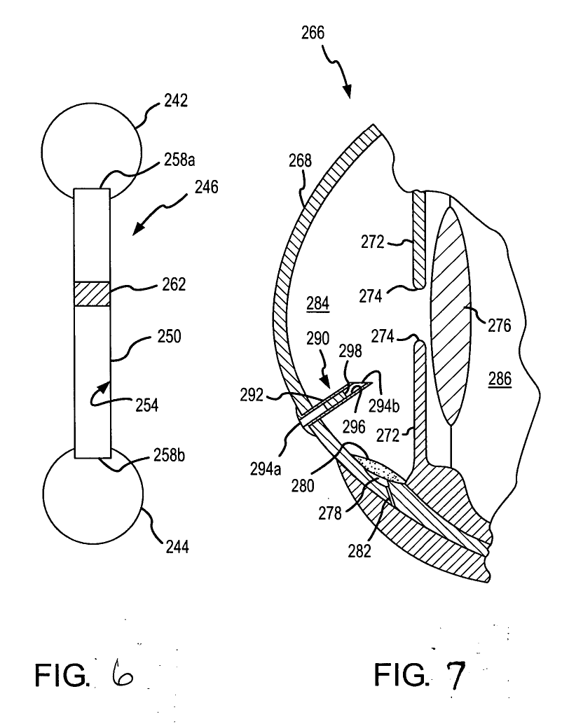

Another embodiment of a glaucoma drainage device, implant, or shunt (“GDD”) is illustrated in FIG. 5 and is identified by reference numeral 406″. The GDD 406″ of FIG. 5 is yet another specific design for the GDD 401 of FIG. 1. The GDD 406″ of FIG. 5 is a variation of the GDD 406′ of FIG. 4, and again may include the suture crossbar 414. Corresponding components between the embodiments of FIGS. 4 and 5 are identified by the same reference numeral. The primary difference between the GDD 406″ of FIG. 5 and the GDD 406′ of FIG. 4 is that multiple flow modules 415 are disposed within the third conduit section 416. Each of the flow modules 415 could be directly disposed in the third conduit section 416, or one or more housings could be used to integrate these structures. Multiple flow modules 415 could also be disposed within the second conduit section 412 of the GDD 406 of FIG. 2.

Generally, the flow modules 415 are disposed in series within the third conduit section 416 of the GDD 406″ of FIG. 5. That is, a flow through the drainage flow path 408′ sequentially passes through the flow modules 415. Each flow module 415 may be in accordance with the above-noted discussion. Typically, one of the flow modules 415 would be in the form of a filter and the other flow module 415 would be in the form of a pressure regulator. The filter flow module 415 could be disposed upstream of the pressure regulator filter module 415, or vice versa. More than two flow modules 415 could be included within the third conduit section 416 as well.

The manner in which the flow modules 415 may be physically replaced has been addressed in relation to the embodiments of FIGS. 2, 4, and 5. There are a number of other points that should be made with regard to replacing flow modules 415 and that apply to each of these embodiments. One is that the first conduit section 409 does not need to be withdrawn from the anterior chamber 284 in order to replace any flow module 415. That is, a surgical procedure should not be required to replace any flow module 415. Moreover, an appropriate anti-bacterial material will typically be applied to the eye 266 and other relevant surfaces before attempting to remove any flow module 415. Finally, the first conduit section 409 may be at least substantially occluded prior to attempting to remove any flow module 415, and the remaining open end (once the flow module(s) 415 is removed) may be plugged in any appropriate manner if desired/required (e.g., the second conduit section 412 could be at least substantially occluded at its second open end 413).

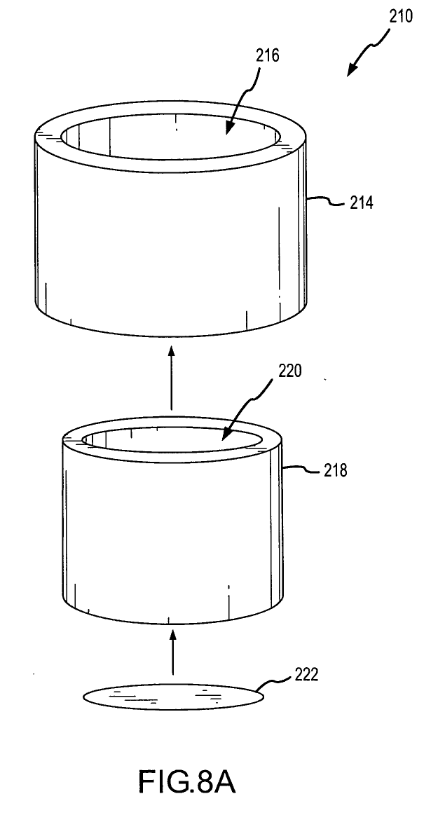

Another example of a system for treating glaucoma is schematically illustrated in FIG. 6. Here, an anterior chamber 242 of a patient's eye (or other body region for that matter—a first body region) is fluidly interconnected with an appropriate drainage area 244 by an implant, shunt, or drainage device 246 (a “glaucoma drainage device” for the specifically noted case). The drainage area 244 may be any appropriate location, such as externally of the eye (e.g., on an exterior surface of the cornea), within the eye (e.g., Schlemm's canal), or within the patient's body in general (a second body region).

Generally, the drainage device 246 includes a conduit 250 having a pair of ends 258a, 258b, with a flow path 254 extending therebetween. The size, shape, and configuration of the conduit 250 may be adapted as desired/required, including to accommodate the specific drainage area 244 being used. Representative configurations for the conduit 250 are disclosed in U.S. Patent Application Publication No. 2003/0212383, as well as U.S. Pat. Nos. 3,788,327; 5,743,868; 5,807,302; 6,626,858; 6,638,239; 6,533,768; 6,595,945; 6,666,841; and 6,736,791, the entire disclosures of which are incorporated by reference in their entirety herein.

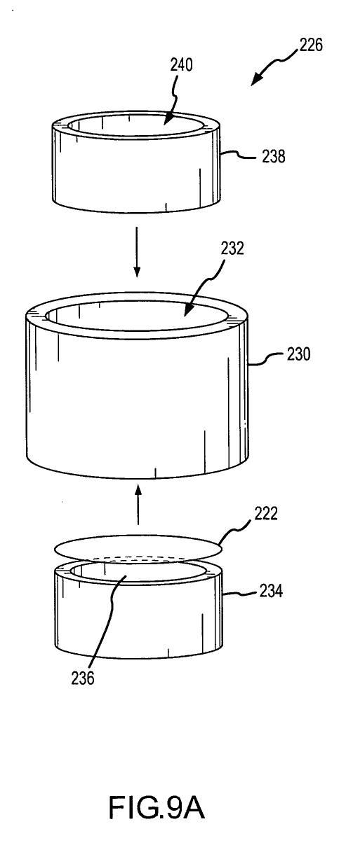

A flow module 262 is disposed within the flow path 254 of the conduit 250. All flow leaving the anterior chamber 242 through the implant 246 is thereby directed through the flow module 262. Similarly, any flow from the drainage area 244 into the drainage device 246 will have to pass through the flow module 262. The flow module 262 may be retained within the conduit 250 in any appropriate manner and at any appropriate location (e.g., it could be disposed on either end 258a, 258b, or any intermediate location therebetween). The flow module 262 may be integrated using one or more housings (e.g., in the manner of any of the flow assemblies 210, 226, or 243 (FIGS. 8A-10B)). Alternatively, the flow module 262 could be directly disposed within the conduit 250 as shown to provide at least one of a filtering function and a pressure-regulating function. Any appropriate coating may be applied to at least those surfaces of the drainage device 246 that would be exposed to biological material/fluids, including without limitation a coating that improves biocompatibility, that makes such surfaces more hydrophilic, and/or that reduces the potential for bio-fouling. In one embodiment, a self-assembled monolayer coating (e.g., poly-ethylene-glycol) is applied in any appropriate manner (e.g., liquid or vapor phase, with vapor phase being the preferred technique) to the noted surfaces.

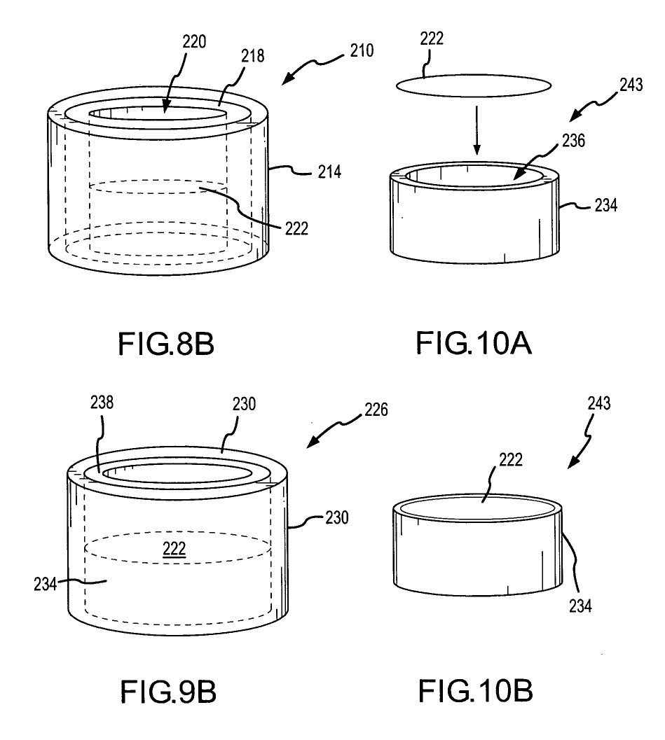

FIG. 7 illustrates a representative embodiment in accordance with FIG. 6. Various portions of the eye 266 are identified in FIG. 7B, including the cornea 268, iris 272, pupil 274, lens 276, anterior chamber 284, vitreous body 286, Schlemm's canal 278, trabecular meshwork 280, and aqueous veins 282. Here, a drainage device, implant, or shunt 290 having an appropriately-shaped conduit 292 is directed through the cornea 268. The conduit 292 may be in any appropriate form, but will typically include at least a pair of ends 294a, 294b, as well as a flow path 296 extending therebetween. End 294a is disposed on the exterior surface of the cornea 268, while end 294b is disposed within the anterior chamber 284 of the eye 266.

A flow module 298 is disposed within the flow path 296 of the conduit 292. All flow leaving the anterior chamber 284 through the drainage device 290 is thereby directed through the flow module 298. Similarly, any flow from the environment back into the drainage device 290 will have to pass through the flow module 298 as well. Preferably, the flow module 298 provides a bacterial filtration function to reduce the potential for developing an infection within the eye when using the drainage device 290. The flow module 298 may be retained within the conduit 292 in any appropriate manner and at any appropriate location (e.g., it could be disposed on either end 294a, 294b, or any an intermediate location therebetween). The flow module 298 may be integrated using one or more housings (e.g., in the manner of any of the flow assemblies 210, 226, or 243 (FIGS. 8A-10B)). Alternatively, the flow module 298 could be directly disposed within the conduit 292 to provide at least one of a filtering function and a pressure-regulating function. Any appropriate coating may be applied to at least those surfaces of the drainage device 290 that would be exposed to biological material/fluids, including without limitation a coating that improves biocompatibility, that makes such surfaces more hydrophilic, and/or that reduces the potential for bio-fouling. In one embodiment, a self-assembled monolayer coating (e.g., poly-ethylene-glycol) is applied in any appropriate manner (e.g., liquid or vapor phase, with vapor phase being the preferred technique) to the noted surfaces.

FIGS. 8A-B schematically represent one embodiment of a flow assembly 210 that may be used for any appropriate application (e.g., the flow assembly 210 may be disposed in a flow of any type, may be used to filter and/or control the flow of a fluid of any type, may be located in a conduit that fluidly interconnects multiple sources of any appropriate type (e.g., between multiple fluid or pressure sources (including where one is the environment), such as a man-made reservoir, a biological reservoir, the environment, or any other appropriate source), or any combination thereof). One example would be to dispose the flow assembly 210 in a conduit extending between the anterior chamber of an eye and a location that is exterior of the cornea of the eye. Another example would be to dispose the flow assembly 210 in a conduit extending between the anterior chamber of an eye and another location that is exterior of the sclera of the eye. Yet another example would be to dispose the flow assembly 210 in a conduit extending between the anterior chamber of an eye and another location within the eye (e.g., into Schlemm's canal) or body. In each of these examples, the conduit would provide an exit path for aqueous humor when installed for a glaucoma patient. That is, each of these examples may be viewed as a way of treating glaucoma or providing at least some degree of control of the intraocular pressure.

Components of the flow assembly 210 include an outer housing 214, an inner housing 218, and a MEMS flow module 222. The position of the MEMS flow module 222 and the inner housing 218 are at least generally depicted within the outer housing 214 in FIG. 8B to show the relative positioning of these components in the assembled condition—not to convey that the outer housing 214 needs to be in the form of a transparent structure. All details of the MEMS flow module 222 and the inner housing 218 are not necessarily illustrated in FIG. 8B.

The MEMS flow module 222 is only schematically represented in FIGS. 8A-B, and provides at least one of a filtering function and a pressure or flow regulation function. The MEMS flow module 222 may be of any appropriate design, size, shape, and configuration, and further may be formed from any material or combination of materials that are appropriate for use by the relevant microfabrication technology. Any appropriate coating or combination of coatings may be applied to exposed surfaces of the MEMS flow module 222 as well. For instance, a coating may be applied to improve the biocompatibility of the MEMS flow module 222, to make the exposed surfaces of the MEMS flow module 222 more hydrophilic, to reduce the potential for the MEMS flow module 222 causing any bio-fouling, or any combination thereof. In one embodiment, a self-assembled monolayer coating (e.g., poly-ethylene-glycol) is applied in any appropriate manner (e.g., liquid or vapor phase, with vapor phase being the preferred technique) to all exposed surfaces of the MEMS flow module 222. The main requirement of the MEMS flow module 222 is that it is a MEMS device.

The primary function of the outer housing 214 and inner housing 218 is to provide structural integrity for the MEMS flow module 222 or to support the MEMS flow module 222, and further to protect the MEMS flow module 222. In this regard, the outer housing 214 and inner housing 218 each will typically be in the form of a structure that is sufficiently rigid to protect the MEMS flow module 222 from being damaged by the forces that reasonably could be expected to be exerted on the flow assembly 210 during its assembly, as well as during use of the flow assembly 210 in the application for which it was designed.

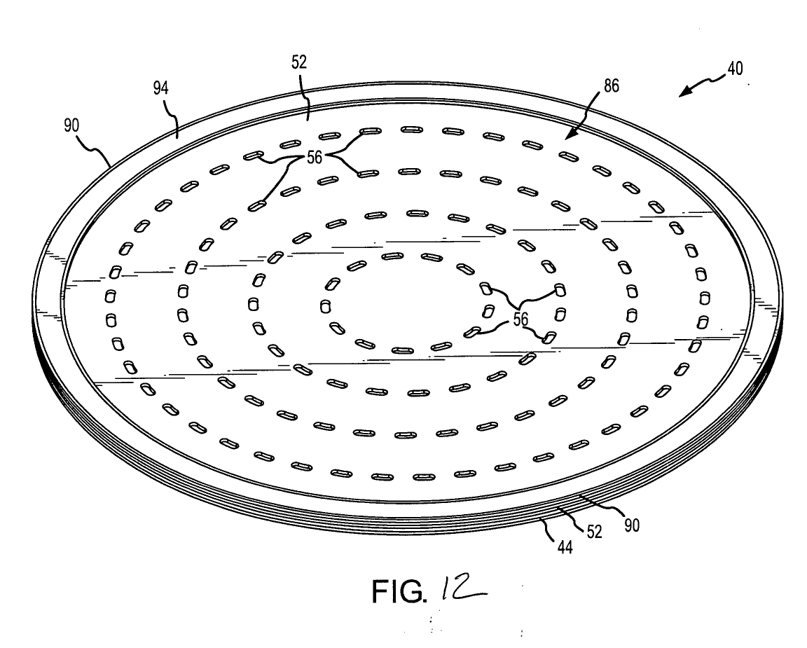

The inner housing 218 includes a hollow interior or a flow path 220 that extends through the inner housing 218 (between its opposite ends in the illustrated embodiment). The MEMS flow module 222 may be disposed within the flow path 220 through the inner housing 218 in any appropriate manner and at any appropriate location within the inner housing 218 (e.g., at any location so that the inner housing 218 is disposed about the MEMS flow module 222). Preferably, the MEMS flow module 222 is maintained in a fixed position relative to the inner housing 218. For instance, the MEMS flow module 222 may be attached or bonded to an inner sidewall or a flange formed on this inner sidewall of the inner housing 218, a press-fit could be provided between the inner housing 218 and the MEMS flow module 222, or a combination thereof. The MEMS flow module 222 also could be attached to an end of the inner housing 218 in the manner of the embodiment of FIGS. 10A-B that will be discussed in more detail below.

The inner housing 218 is at least partially disposed within the outer housing 214 (thereby encompassing having the outer housing 214 being disposed about the inner housing 218 along the entire length of the inner housing 218, or only along a portion of the length of the inner housing 218). In this regard, the outer housing 214 includes a hollow interior 216 for receiving the inner housing 218, and possibly to provide other appropriate functionality (e.g., a flow path fluidly connected with the flow path 220 through the inner housing 218). The outer and inner sidewalls of the outer housing 214 may be cylindrical or of any other appropriate shape, as may be the outer and inner sidewalls of the inner housing 218. The inner housing 218 may be retained relative to the outer housing 214 in any appropriate manner. For instance, the inner housing 218 may be attached or bonded to an inner sidewall of the outer housing 214, a press-fit could be provided between the inner housing 218 and the outer housing 214, a shrink fit could be provided between the outer housing 214 and the inner housing 218, or a combination thereof.

The inner housing 218 is likewise only schematically represented in FIGS. 8A-B, and it may be of any appropriate shape/configuration, of any appropriate size, and formed from any material or combination of materials (e.g., polymethylmethacrylate (PMMA), ceramics, silicon, titanium, and other implantable metals and plastics). Typically its outer contour will be adapted to match the inner contour of the outer housing 214 in which it is at least partially disposed. In one embodiment, the illustrated cylindrical configuration for the inner housing 218 is achieved by cutting an appropriate length from hypodermic needle stock. The inner housing 218 also may be microfabricated into the desired/required shape (e.g., using at least part of a LIGA process). However, any way of making the inner housing 218 may be utilized. It should also be appreciated that the inner housing 218 may include one or more coatings as desired/required as well (e.g., an electroplated metal; a coating to improve the biocompatibility of the inner housing 218, to make the exposed surfaces of the inner housing 218 more hydrophilic, to reduce the potential for the inner housing 218 causing any bio-fouling, or any combination thereof). In one embodiment, a self-assembled monolayer coating (e.g., poly-ethylene-glycol) is applied in any appropriate manner (e.g., liquid or vapor phase, with vapor phase being the preferred technique) to all exposed surfaces of the inner housing 218.

The outer housing 214 likewise is only schematically represented in FIGS. 8A-B, and it may be of any appropriate shape/configuration, of any appropriate size, and formed from any material or combination of materials (e.g., polymethylmethacrylate (PMMA), ceramics, silicon, titanium, and other implantable metals and plastics). Typically its outer contour will be adapted to match the inner contour of the housing or conduit in which it is at least partially disposed or otherwise mounted. The outer housing 214 also may be microfabricated into the desired/required shape (e.g., using at least part of a LIGA process). However, any way of making the outer housing 214 may be utilized. It should also be appreciated that the outer housing 214 may include one or more coatings as desired/required as well (e.g., an electroplated metal; a coating to improve the biocompatibility of the outer housing 214, to make the exposed surfaces of the outer housing 214 more hydrophilic, to reduce the potential for the outer housing 214 causing any bio-fouling, or any combination thereof). In one embodiment, a self-assembled monolayer coating (e.g., poly-ethylene-glycol) is applied in any appropriate manner (e.g., liquid or vapor phase, with vapor phase being the preferred technique) to all exposed surfaces of the outer housing 214.

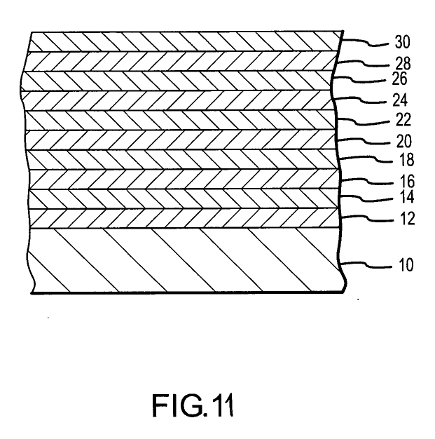

Another embodiment of a flow assembly is illustrated in FIGS. 9A-B (only schematic representations), and is identified by reference numeral 226. The flow assembly 226 may be used for any appropriate application (e.g., the flow assembly 226 may be disposed in a flow of any type, may be used to filter and/or control the flow of a fluid of any type, may be located in a conduit that fluidly interconnects multiple sources of any appropriate type (e.g., multiple fluid or pressure sources (including where one is the environment), such as a man-made reservoir, a biological reservoir, the environment, or any other appropriate source), or any combination thereof). The above-noted applications for the flow assembly 210 are equally applicable to the flow assembly 226. The types of coatings discussed above in relation to the flow assembly 210 may be used by the flow assembly 226 as well.

Components of the flow assembly 226 include an outer housing 230, a first inner housing 234, a second inner housing 238, and the MEMS flow module 222. The MEMS flow module 222 and the inner housings 234, 238 are at least generally depicted within the outer housing 230 in FIG. 9B to show the relative positioning of these components in the assembled condition—not to convey that the outer housing 230 needs to be in the form of a transparent structure. All details of the MEMS flow module 222 and the inner housings 234, 238 are not necessarily illustrated in FIG. 9B.

The primary function of the outer housing 230, first inner housing 234, and second inner housing 238 is to provide structural integrity for the MEMS flow module 222 or to support the MEMS flow module 222, and further to protect the MEMS flow module 222. In this regard, the outer housing 230, first inner housing 234, and second inner housing 238 each will typically be in the form of a structure that is sufficiently rigid to protect the MEMS flow module 222 from being damaged by the forces that reasonably could be expected to be exerted on the flow assembly 226 during its assembly, as well as during use of the flow assembly 226 in the application for which it was designed.

The first inner housing 234 includes a hollow interior or a flow path 236 that extends through the first inner housing 234. Similarly, the second inner housing 238 includes a hollow interior or a flow path 240 that extends through the second inner housing 238. The first inner housing 234 and the second inner housing 238 are disposed in end-to-end relation, with the MEMS flow module 222 being disposed between adjacent ends of the first inner housing 234 and the second inner housing 238. As such, a flow progressing through the first flow path 236 to the second flow path 240, or vice versa, passes through the MEMS flow module 222.

Preferably, the MEMS flow module 222 is maintained in a fixed position relative to each inner housing 234, 238, and its perimeter does not protrude beyond the adjacent sidewalls of the inner housings 234, 238 in the assembled and joined condition. For instance, the MEMS flow module 222 may be bonded to at least one of, but more preferably both of, the first inner housing 234 (more specifically one end thereof) and the second inner housing 238 (more specifically one end thereof) to provide structural integrity for the MEMS flow module 222 (e.g., using cyanoacrylic esters, thermal bonding, UV-curable epoxies, or other epoxies). Another option would be to fix the position the MEMS flow module 222 in the flow assembly 226 at least primarily by fixing the position of each of the inner housings 234, 238 relative to the outer housing 230 (i.e., the MEMS flow module 222 need not necessarily be bonded to either of the housings 234, 238). In one embodiment, an elastomeric material may be disposed between the MEMS flow module 222 and the first inner housing 234 to allow the first inner housing 234 with the MEMS flow module 222 disposed thereon to be pushed into the outer housing 230 (e.g., the elastomeric material is sufficiently “tacky” to at least temporarily retain the MEMS flow module 222 in position relative to the first inner housing 234 while being installed in the outer housing 230). The second inner housing 238 also may be pushed into the outer housing 230 (before, but more likely after, the first inner housing 234 is disposed in the outer housing 230) to “sandwich” the MEMS flow module 222 between the inner housings 234, 238 at a location that is within the outer housing 230 (i.e., such that the outer housing 230 is disposed about MEMS flow module 222). The MEMS flow module 222 would typically be contacted by both the first inner housing 234 and the second inner housing 238 when disposed within the outer housing 230. Fixing the position of each of the first inner housing 234 and the second inner housing 238 relative to the outer housing 230 will thereby in effect fix the position of the MEMS flow module 222 relative to the outer housing 230. Both the first inner housing 234 and second inner housing 238 are at least partially disposed within the outer housing 230 (thereby encompassing the outer housing 230 being disposed about either or both housings 234, 238 along the entire length thereof, or only along a portion of the length of thereof), again with the MEMS flow module 222 being located between the adjacent ends of the first inner housing 234 and the second inner housing is 238. In this regard, the outer housing 230 includes a hollow interior 232 for receiving at least part of the first inner housing 234, at least part of the second inner housing 238, and the MEMS flow module 222 disposed therebetween, and possibly to provide other appropriate functionality (e.g., a flow path fluidly connected with the flow paths 236, 240 through the first and second inner housings 234, 238, respectively). The outer and inner sidewalls of the outer housing 230 may be cylindrical or of any other appropriate shape, as may be the outer and inner sidewalls of the inner housings 234, 238. Both the first inner housing 234 and the second inner housing 238 may be secured to the outer housing 230 in any appropriate manner, including in the manner discussed above in relation to the inner housing 218 and the outer housing 214 of the embodiment of FIGS. 8A-B.

Each inner housing 234, 238 is likewise only schematically represented in FIGS. 9A-B, and each may be of any appropriate shape/configuration, of any appropriate size, and formed from any material or combination of materials in the same manner as the inner housing 218 of the embodiment of FIGS. 8A-B. Typically the outer contour of both housings 234, 238 will be adapted to match the inner contour of the outer housing 230 in which they are at least partially disposed. In one embodiment, the illustrated cylindrical configuration for the inner housings 234, 238 is achieved by cutting an appropriate length from hypodermic needle stock. The inner housings 234, 238 each also may be microfabricated into the desired/required shape (e.g., using at least part of a LIGA process). However, any way of making the inner housings 234, 238 may be utilized. It should also be appreciated that the inner housings 234, 238 may include one or more coatings as desired/required as well in accordance with the foregoing.

The outer housing 230 is likewise only schematically represented in FIGS. 9A-B, and it may be of any appropriate shape/configuration, of any appropriate size, and formed from any material or combination of materials in the same manner as the outer housing 214 of the embodiment of FIGS. 8A-B. Typically the outer contour of the outer housing 230 will be adapted to match the inner contour of the housing or conduit in which it is at least partially disposed or otherwise mounted. The outer housing 230 may be microfabricated into the desired/required shape (e.g., using at least part of a LIGA process). However, any way of making the outer housing 230 may be utilized. It should also be appreciated that the outer housing 230 may include one or more coatings as desired/required in accordance with the foregoing.

Another embodiment of a flow assembly is illustrated in FIGS. 10A-B (only schematic representations), and is identified by reference numeral 243. The flow assembly 243 may be used for any appropriate application (e.g., the flow assembly 243 may be disposed in a flow of any type, may be used to filter and/or control the flow of a fluid of any type, may be located in a conduit that fluidly interconnects multiple sources of any appropriate type (e.g., between multiple fluid or pressure sources, such as a man-made reservoir, a biological reservoir, the environment, or any other appropriate source), or any combination thereof). Components of the flow assembly 243 include the above-noted housing 234 and the MEMS flow module 222 from the embodiment of FIGS. 9A-B. In the case of the flow assembly 243, the MEMS flow module 222 is attached or bonded to one end of the housing 234 (e.g., using cyanoacrylic esters, thermal bonding, UV-curable epoxies, or other epoxies). The flow assembly 243 may be disposed within an outer housing in the manner of the embodiments of FIGS. 8A-9B, or could be used “as is.” The above-noted applications for the flow assembly 210 are equally applicable to the flow assembly 243. The types of coatings discussed above in relation to the flow assembly 210 may be used by the flow assembly 243 as well.

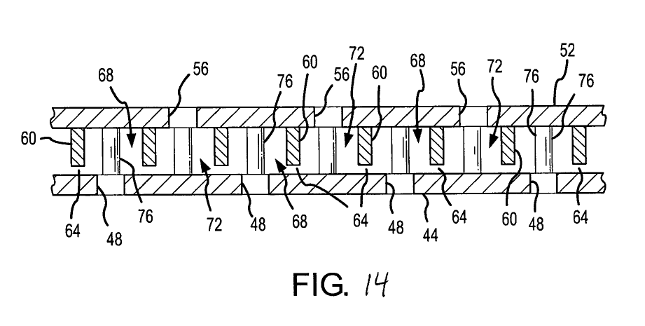

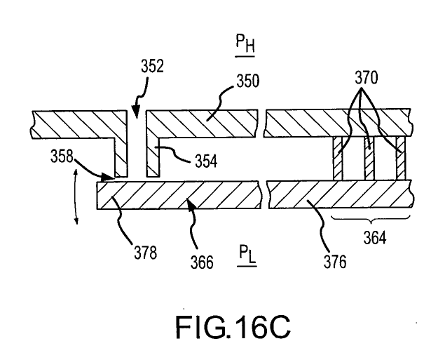

Various MEMS devices that provide at least one of a filtering function and a pressure-regulating function are disclosed in the patent applications noted above in the “Cross-Reference to Related Applications” section of this patent application. Each of these MEMS devices could be used by the GDDs 6, 6′, and 6″ of FIGS. 2, 4, and 5 (as a flow module 415) to realize the above-noted characteristics/parameters for the GDD 1 of FIG. 1. That is, each of these MEMS devices will accommodate a high flow rate through a relatively small cross-sectional area (a flow rate of at least about 0.15 microliters/minute/mm²/mm-Hg, and the other higher flow rates noted above). Each of these MEMS devices may be fabricated to filter particles having a minimum dimension of at least a certain magnitude, may be fabricated to target filtering specific organisms (e.g., pseudomonas aeruginosa, staphylococcus aureus, brevundimonas diminuta), or both. Each of these MEMS devices could also be used as the MEMS flow module 222 in the case of the flow assemblies 210, 226, and 243 of FIGS. 8A-10B. Finally, each of these MEMS devices could be used as the flow module 262 for the drainage device 246 of FIG. 6 or could be used by or as the flow module 298 for the drainage device 290 of FIG. 7. One of these MEMS devices that primarily provides a filtering function will be described herein (MEMS flow module 40 of FIGS. 12-15), and one of these MEMS devices that provides at least a pressure-regulating function will also be described herein (MEMS flow module 340 of FIGS. 16A-C).

Generally, the MEMS devices in the above-noted patent applications are microfabricated. There are a number of microfabrication technologies that are commonly characterized as “micromachining,” including without limitation LIGA (Lithographie, Galvonoformung, Abformung), SLIGA (sacrificial LIGA), bulk micromachining, surface micromachining, micro electrodischarge machining (EDM), laser micromachining, 3-D stereolithography, and other techniques. Hereafter, the term “MEMS device,” “microfabricated device,” or the like means any such device that is fabricated using a technology that allows realization of a feature size of 10 microns or less. Any appropriate microfabrication technology or combination of microfabrication technologies may be used to fabricate the noted various MEMS devices.

Surface micromachining is currently the preferred fabrication technique for the various MEMS devices disclosed by the above-noted patent applications. One particularly desirable surface micromachining technique is described in U.S. Pat. No. 6,082,208, that issued Jul. 4, 2000, that is entitled “Method For Fabricating Five-Level Microelectromechanical Structures and Microelectromechanical Transmission Formed,” and the entire disclosure of which is incorporated by reference in its entirety herein. Surface micromachining generally entails depositing alternate layers of structural material and sacrificial material using an appropriate substrate (e.g., a silicon wafer) which functions as the foundation for the resulting microstructure. Various patterning operations (collectively including masking, etching, and mask removal operations) may be executed on one or more of these layers before the next layer is deposited so as to define the desired microstructure. After the microstructure has been defined in this general manner, all or a portion of the various sacrificial layers are removed by exposing the microstructure and the various sacrificial layers to one or more etchants. This is commonly called “releasing” the microstructure.

The term “sacrificial layer” as used herein means any layer or portion thereof of any surface micromachined microstructure that is used to fabricate the microstructure, but which does not generally exist in the final configuration (e.g., sacrificial material may be encased by a structural material at one or more locations for one or more purposes, and as a result this encased sacrificial material is not removed by the release). Exemplary materials for the sacrificial layers described herein include undoped silicon dioxide or silicon oxide, and doped silicon dioxide or silicon oxide (“doped” indicating that additional elemental materials are added to the film during or after deposition). The term “structural layer” as used herein means any other layer or portion thereof of a surface micromachined microstructure other than a sacrificial layer and a substrate on which the microstructure is being fabricated. Exemplary materials for the structural layers described herein include doped or undoped polysilicon and doped or undoped silicon. Exemplary materials for the substrates described herein include silicon. The various layers described herein may be formed/deposited by techniques such as chemical vapor deposition (CVD) and including low-pressure CVD (LPCVD), atmospheric-pressure CVD (APCVD), and plasma-enhanced CVD (PECVD), thermal oxidation processes, and physical vapor deposition (PVD) and including evaporative PVD and sputtering PVD, as examples.

In more general terms, surface micromachining can be done with any suitable system of a substrate, sacrificial film(s) or layer(s) and structural film(s) or layer(s). Many substrate materials may be used in surface micromachining operations, although the tendency is to use silicon wafers because of their ubiquitous presence and availability. The substrate is essentially a foundation on which the microstructures are fabricated. This foundation material must be stable to the processes that are being used to define the microstructure(s) and cannot adversely affect the processing of the sacrificial/structural films that are being used to define the microstructure(s). With regard to the sacrificial and structural films, the primary differentiating factor is a selectivity difference between the sacrificial and structural films to the desired/required release etchant(s). This selectivity ratio may be on the order of about 10:1, and is more preferably several hundred to one or much greater, with an infinite selectivity ratio being most preferred. Examples of such a sacrificial film/structural film system include: various silicon oxides/various forms of silicon; poly germanium/poly germanium-silicon; various polymeric films/various metal films (e.g., photoresist/aluminum); various metals/various metals (e.g., aluminum/nickel); polysilicon/silicon carbide; silicone dioxide/polysilicon (i.e., using a different release etchant like potassium hydroxide, for example). Examples of release etchants for silicon dioxide and silicon oxide sacrificial materials are typically hydrofluoric (HF) acid based (e.g., concentrated HF acid, which is actually 49 wt % HF acid and 51 wt % water; concentrated HF acid further diluted with water; buffered HF acid (HF acid and ammonium fluoride)).

The microfabrication technology described in the above-noted '208 patent uses a plurality of alternating structural layers (e.g., polysilicon and therefore referred to as “P” layers herein) and sacrificial layers (e.g., silicon dioxide, and therefore referred to as “S” layers herein). The nomenclature that is commonly used to describe the various layers in the microfabrication technology described in the above-noted '208 patent will also be used herein.

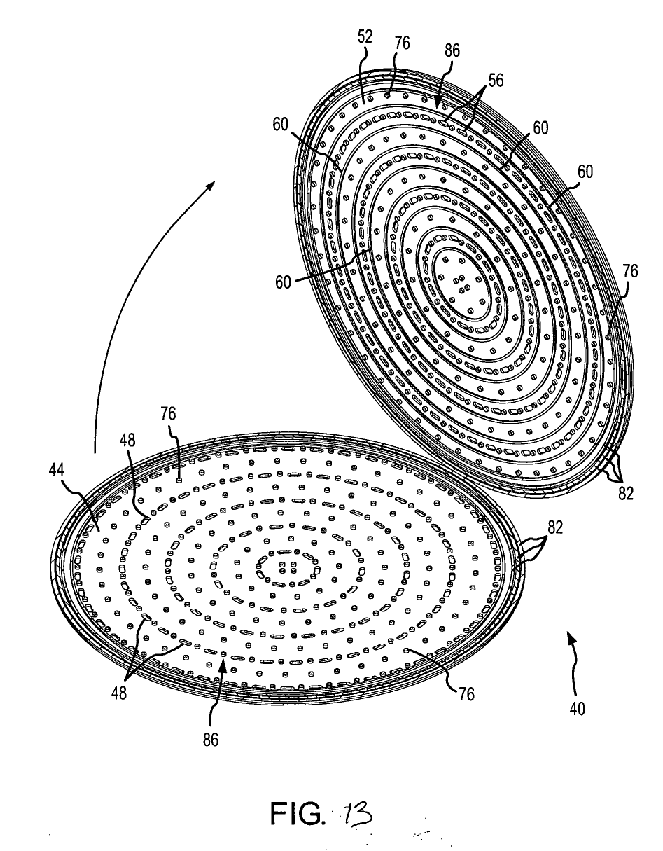

FIG. 11 generally illustrates one embodiment of layers on a substrate 10 that is appropriate for surface micromachining and in accordance with the nomenclature commonly associated with the '208 patent. Each of these layers will typically have a thickness of no more than about 10 microns, and more typically a thickness within a range of about 1 micron to about 3 microns. Progressing away from the substrate 10, the various layers are: a dielectric layer 12 (there may be an intermediate oxide layer between the dielectric layer 12 and the substrate 10 as well, which is not shown); a P₀ layer 14 (a first fabrication level); an S₁ layer 16; a P₁ layer 18 (a second fabrication level); an S₂ layer 20; a P₂ layer 22 (a third fabrication level); an S₃ layer 24; a P₃ layer 26 (a fourth fabrication level); an S₄ layer 28; and a P₄ layer 30 (a fifth fabrication level). In some cases, the S₂ layer 20 may be removed before the release such that the P₂ layer 22 is deposited directly on the P₁ layer 18, and such will hereafter be referred to as a P₁/P₂ layer. It should also be appreciated that one or more other layers may be deposited on the P₄ layer 30 after the formation thereof and prior to the release, where the entirety of the S₁ layer 16, S₂ layer 20, S₃ layer 24, and S₄ layer 28 may be removed (although portions of one or more of these layers may be retained for one or more purposes if properly encased so as to be protected from the release etchant). It should also be appreciated that adjacent structural layers may be structurally interconnected by forming cuts or apertures through the entire thickness of a particular sacrificial layer before depositing the next structural layer. In this case, the structural material will not only be deposited on the upper surface of the particular sacrificial layer, but will be deposited in these cuts or apertures as well (and will thereby interconnect a pair of adjacent, spaced, structural layers).

One embodiment of a MEMS flow module is illustrated in FIGS. 12-15, is identified by reference numeral 40, and provides at least a filtering function. Components of the MEMS flow module 40 include a first plate 44 (e.g., fabricated in at least P₂ layer 22; fabricated in a combination of P₂ layer 22 that is disposed directly on P₁ layer 18), a second plate 52 (e.g., fabricated in P₃ layer 26), and a plurality of filtering walls 60 (e.g., fabricated in P₃ layer 26). The first plate 44 includes a plurality of first flow ports 48 that extend completely through the first plate 44, while the second plate 52 includes a plurality of second flow ports 56 that extend completely through the second plate 52. Both the first flow ports 48 and the second flow ports 56 may be of any appropriate size, shape, and/or configuration.

The first plate 44 and the second plate 52 of the MEMS flow module 40 may be maintained in at least a substantially fixed position relative to each other. In this regard, a plurality of structural interconnects 76 extend between and structurally interconnect the first plate 44 and the second plate 52 so as to maintain the same in spaced relation. Each structural interconnect 76 may be of any appropriate size, shape, and/or configuration (e.g., in the form of a column or post, as shown), and the plurality of structural interconnects 76 may be disposed in any appropriate arrangement.

Perimeter portions of the first plate 44 and the second plate 52 also may be structurally interconnected by one or more annular structural interconnects 82. “Annular” only means that the structural interconnects 82 extend a full 360° about a common point, and does not limit the annular structural interconnects 82 to having a circular configuration. Representative annular configurations for the annular structural interconnects 82 include circular, square-shaped, rectangular-shaped, elliptical-shaped or the like. Each annular structural interconnect 82 also provides a lateral or radial seal function by reducing the potential for a flow exiting the MEMS flow module 40 from the space between the first plate 44 and the second plate 52. Utilizing multiple, laterally or radially-spaced annular structural interconnects 82 thereby provides redundant lateral or radial seals.

The MEMS flow module 40 accommodates filtering of a bidirectional flow, or a flow through the MEMS flow module 40 in either of two general directions. That is, a flow may be directed into the MEMS flow module 40 through one or more of the first flow ports 48 of the first plate 44 and may exit the MEMS flow module 40 through one or more of the second flow ports 56 of the second plate 52. A flow may also be directed into the MEMS flow module 40 through one or more of the second flow ports 56 of the second plate 52 and may exit the MEMS flow module 40 through one or more of the first flow ports 48 of the first plate 44. Regardless of the direction of flow through the MEMS flow module 40, this flow is filtered by the first plate 44 cooperating with a plurality of the filtering walls 60 of the MEMS flow module 40.

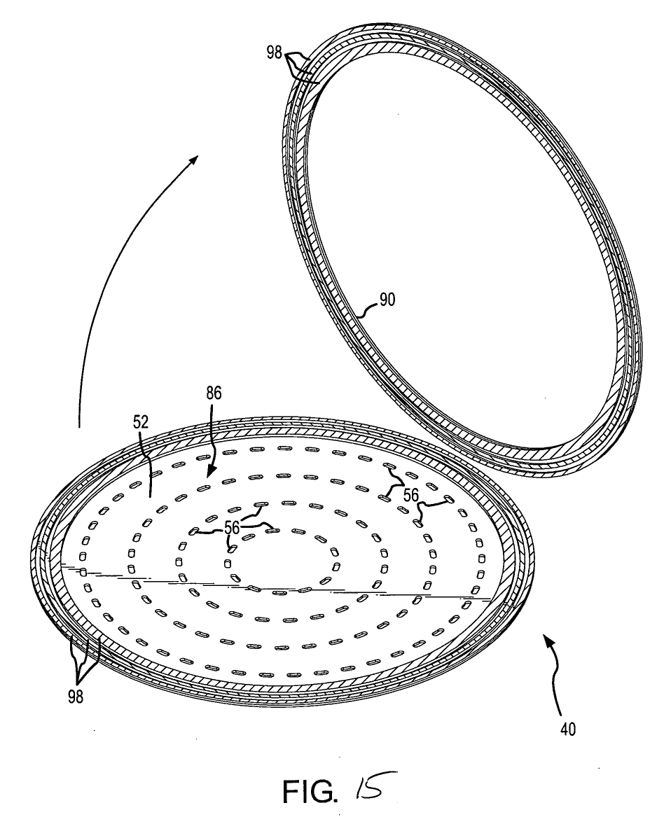

Each filtering wall 60 extends from the second plate 52 toward, but not to, the first plate 44 (the cross-sectional view in FIG. 13 is taken between the first plate 44 and the second plate 52 along a reference plane that extends through the space between the first plate 44 and the end of each of the filtering walls 60). That is, each filtering wall 60 terminates prior to reaching the first plate 44. Any appropriate number of filtering walls 60 may be utilized by the MEMS flow module 40.

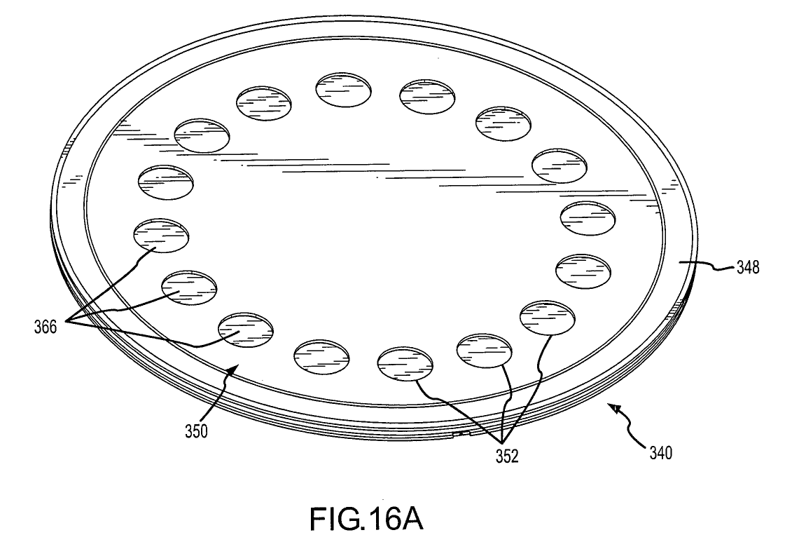

The space between the end of each filtering wall 60 and the first plate 44 is identified as a filter trap 64 (FIG. 14) and is dimensioned so as to filter objects of a certain size or larger. The height of each filter trap 64 (the distance between the end of each filtering wall 60 and the first plate 44) is no more than about 0.4 microns in one embodiment, is about 0.2 to about 0.3 microns in one embodiment, and is no more than about 0.1 microns in yet another embodiment. The size of each filter trap 64 may be established at any appropriate value. Although each filter trap 64 may be of at least substantially the same size, such need not always be the case.

Each filtering wall 60 is annular in that each filtering wall 60 extends a full 360° about a reference point. Although a circular annular configuration is preferred for the filtering walls 60, other annular configurations could be utilized as well (e.g., square-shaped, rectangular-shaped, elliptical-shaped). In addition, one or more filtering walls 60 could be replaced by a plurality of appropriately spaced filtering wall segments (not shown). In any case, the filtering walls 60 are disposed in a desired arrangement that is believed to accommodate a desirably high flow rate through the MEMS flow module 40. In this regard, the various filtering walls 60 are at least generally concentrically disposed about a common center or point (i.e., each being disposed at a different radius from a common center or point). Although it is preferred for the filtering walls 60 to be equally spaced in this concentric arrangement, such is not necessarily required.

The various first flow ports 48, the various second flow ports 56, and the various filtering walls 60 are located in what may be characterized as a filtering region 86 of the MEMS flow module 40. The filtering region 86 is located inwardly of the innermost annular structural interconnect 82 between the first plate 44 and the second plate 52. Generally, the first flow ports 48 through the first plate 44 and the second flow ports 56 through the second plate 52 are arranged such that: 1) any flow entering the MEMS flow module 40 through any first flow port 48 will flow through a filter trap 64 prior to exiting the MEMS flow module 40 through any second flow port 56; and 2) any flow entering the MEMS flow module 40 through any second flow port 56 will flow through a filter trap 64 prior to exiting the MEMS flow module 40 through any first flow port 48.

The space between each adjacent pair of filtering walls 60 is accessed by either one or more first flow ports 48 or one or more second flow ports 56 in order to force a flow through at least one filter trap 64 in the case of the MEMS flow module 40. Stated another way, the MEMS flow module 40 may be characterized as including a plurality of first flow port chambers 68 and a plurality of the second flow port chambers 72 (e.g., FIG. 14). Each first flow port chamber 68 and each second flow port chamber 72 is defined by a spacing between the first plate 44 and the second plate 52 in a first dimension, and is defined by a spacing between adjacent filtering walls 60 in a second dimension that is orthogonal to the first dimension. Only first flow ports 48 directly fluidly communicate with each first flow port chamber 68—no second flow port 56 can access a first flow port chamber 68 without first passing through a filter trap 64. Similarly, only second flow ports 56 directly fluidly communicate with each second flow port chamber 72—no first flow port 48 can access a second flow port chamber 72 without first passing through a filter trap 64.

The above-noted first flow port chambers 68 and the second flow port chambers 72 are disposed in alternating relation to force all flow through at least one filter trap 64. For instance, a flow entering the MEMS flow module 40 through one or more first flow ports 48 of a particular first flow port chamber 68 would need to flow through at least one filter trap 64 before entering any second flow port chamber 72, such that the flow could then exit the MEMS flow module 40 through one or more second flow ports 56 associated with this particular second flow port chamber 72. Similarly, a flow entering the MEMS flow module 40 through one or more second flow ports 56 of a particular second flow port chamber 72 would need to flow through at least one filter trap 64 before entering any first flow port chamber 68, such that the flow could then exit the MEMS flow module 40 through one or more first flow ports 48 associated with this particular first flow port chamber 68.

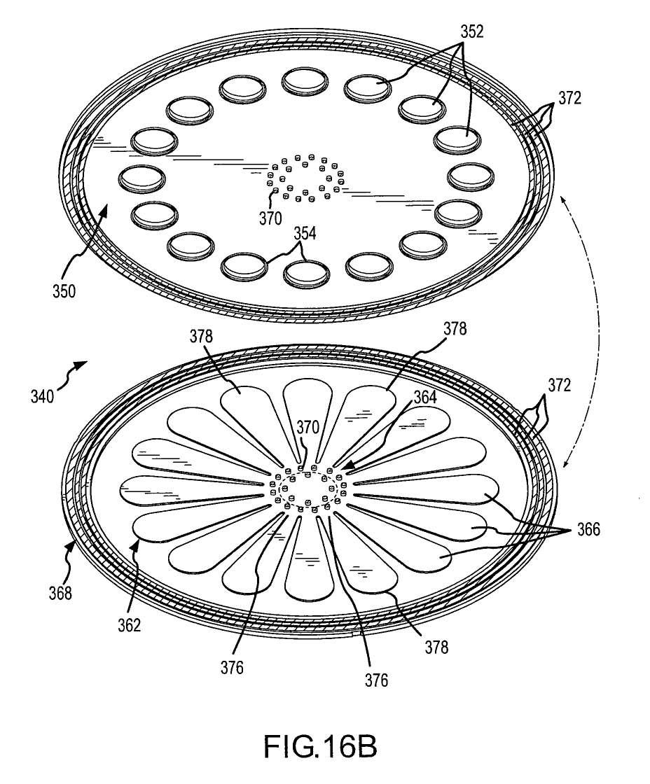

The MEMS flow module 40 could simply be in the form of the above-noted first plate 44, the second plate 52, and the filtering walls 60. However, it may be desirable to include one or more additional structures for one or more purposes. In this regard, the MEMS flow module 40 also may include an annular support 90 (e.g., fabricated in P₄ layer 30) that is spaced from and interconnected with a perimeter portion of the second plate 52 by a one or more annular structural interconnects 98 (FIGS. 12 and 15). Any appropriate number of annular structural interconnects 98 may be utilized. The second plate 52 is thereby “sandwiched” between the first plate 44 and the annular support 90. This configuration may enhance the rigidity of the MEMS flow module 40, or at least enhance an interface between the MEMS flow module 40 and one or more housings that may be utilized to contain/support the MEMS flow module 40 for a particular application. The annular support 90 could also be deposited directly on the second plate 52.

The MEMS flow module 40 may further include a ring 94 (FIG. 12) that is fixedly positioned on the surface of the annular support 90 that is opposite that which interfaces with the second plate 52. This ring 94 may be an appropriate metal that is attached to or formed on the annular support 90 after the MEMS flow module 40 has been fabricated, or may in fact be formed by surface micromachining as well (e.g., from another structural level). Generally, the ring 94 may provide a desired interface with a housing or other structure that incorporates the MEMS flow module 40. It should be appreciated that one or more additional plates with flow ports extending therethrough could be interconnected with or formed directly on either the first plate 44 or the second plate 52, and for any desired purpose.

The MEMS flow module 40 may be used for any appropriate application. One particularly desirable application is to use the MEMS flow module 40 in an implant that addresses the pressure in the anterior chamber of a patient's eye that is diseased. The size of the filter traps 64 may be selected to balance the desire to at least generally mimic the flow of aqueous humor out of the anterior chamber of a patient's eye through the eye's canal of Schlemm (e.g., provide a sufficient “back pressure”), along with the desire to be able to accommodate an increase in flow of aqueous humor out of the anterior chamber of the eye so relieve at least certain increases in the intraocular pressure in a desired manner.

Surface micromachining is the preferred technology for fabricating the above-described MEMS flow module 40. In this regard, the above-noted MEMS flow module 40 may be suspended above the substrate 10 after the release by one or more suspension tabs that are disposed about the perimeter of the MEMS flow module 40, that engage an appropriate portion of the MEMS flow module 40, and that are anchored to the substrate 10. These suspension tabs may be fractured or broken (e.g., by application of the mechanical force; electrically, such as by directing an appropriate current through the suspension tabs) to structurally disconnect the MEMS flow module 40 from the substrate 10. One or more motion limiters may be fabricated and disposed about the perimeter of the MEMS flow module 40 as well to limit the amount that the MEMS flow module 40 may move in the lateral or radial dimension after the suspension tabs have been fractured and prior to retrieving the disconnected MEMS flow module 40. Representative suspension tabs and motion limiters are disclosed in commonly owned U.S. patent application Ser. No. 11/048,195.

The MEMS flow module 40 described herein may be fabricated in at least two different levels that are spaced from each other (hereafter a first fabrication level and a second fabrication level). Generally, that MEMS flow module 40 again includes the first plate 44 and the second plate 52 that are disposed in spaced relation, with a plurality of filtering walls 60 extending from the second plate 52 at least toward the first plate 44. The first plate 44 and its various first flow ports 48 may be fabricated in a first fabrication level, while the second plate 52 and its various second flow ports 56 and filtering walls 60 may be fabricated in a second fabrication level. It should be appreciated that the characterization of the first plate 44 being in a “first fabrication level” and the second plate 52 and filtering walls 60 being in the “second fabrication level” by no means requires that the first fabrication level be that which is deposited “first”, and that the second fabrication level be that which is deposited “second.” Moreover, it does not require that the first fabrication level and the second fabrication level be immediately adjacent.

One or both of the first plate 44 and that second plate 52 each may exist in a single fabrication level or may exist in multiple fabrication levels. “Fabrication level” corresponds with what may be formed by a deposition of a structural material before having to form any overlying layer of a sacrificial material (e.g., from a single deposition of a structural layer or film). In the above-noted first instance, a deposition of a structural material in a single fabrication level may define an at least generally planar layer. Another option regarding the first instance would be for the deposition of a structural material in a single fabrication level to define an at least generally planar portion, plus one or more structures that extend down toward, but not to, the underlying structural layer at the underlying fabrication level (e.g., the second plate 52 with the various filtering walls 60 extending downwardly therefrom, the fabrication of which is discussed in more detail below). In either situation and prior to the release, in at least some cases there will be at least some thickness of sacrificial material disposed between the entirety of the structures in adjacent fabrication levels (e.g., between the distal end of the filtering walls 60 and the first plate 44; between the first plate 44 and the second plate 52).

In the above-noted second instance, two or more structural layers or films from adjacent fabrication levels could be disposed in direct interfacing relation (e.g., one directly on the other). Over the region that is to define a pair of plates, this would require removal of at least some of the sacrificial material that is deposited on the structural material at one fabrication level before depositing the structural material at the next fabrication level (e.g., the annular support 90 could be deposited directly on a perimeter portion of the second plate 52, as previously noted). Another option regarding the above-noted second instance would be to maintain the separation between structural layers or films in different fabrication levels for a pair of plates, but provide an appropriate structural interconnection therebetween (e.g., a plurality of columns, posts, or the like extending between adjacent structural layers or films in different, spaced fabrication levels). For instance and as described above, the first plate 44 and the second plate 52 are disposed in spaced relation, but perimeter portions thereof are interconnected by the annular structural interconnects 82. The first plate 44 and the second plate 52 are also maintained in spaced relation by the structural interconnects 76 disposed within the filtering region 86. The structural interconnects 76, the annular structural interconnects 82, the second plate 52, and the filtering walls 60 may be fabricated in a common fabrication level.

With further regard to fabricating the MEMS flow module 40 at least in part by surface micromachining, each component thereof (including without limitation the first plate 44 and/or the second plate 52) may be fabricated in a structural layer or film at a single fabrication level (e.g., in P₁ layer 18; in P₂ layer 22; in P₃ layer 26; in P₄ layer 30 (FIG. 11 discussed above)). One example of fabricating the MEMS flow module 40 by surface micromachining would be to fabricate the first plate 44 at least in the P₂ layer 22 (possibly in the P₁ layer 18 as well, where the P₂ layer 22 is deposited directly on at least part of the P₁ layer 18). After at least the P₂ layer 22 has been patterned to define the perimeter of the first plate 44 and the various first flow ports 48 that extend through the first plate 44, the S₃ layer 24 may be deposited on top of the first plate 44 and into the first flow ports 48. Annular first troughs may then be patterned in the S₃ layer 24 to coincide with the location of the filtering walls 60, where these first troughs extend all the way down to the P₂ layer 22. Sacrificial material may be deposited in the bottom of these annular first troughs (the thickness of which will define the spacing between the ends of the filtering walls 60 and the first plate 44, or stated another way the height of the filter traps 64). The thickness of this deposition may be controlled with reasonable precision, or definable at small dimensions, to define a filter trap 64 of a desired height. In this regard, the size of the largest filter trap gap 64 should be no more than about 105% of the size of the smallest filter trap gap 64. One embodiment has the thickness of this deposition being no more than about 0.4 microns. Another embodiment has the thickness of this deposition being about 0.2 to about 0.3 microns. Yet another embodiment has the thickness of this deposition being about 0.1 microns or even less.

Annular second troughs may also be patterned in the above-noted S₃ layer 24 to coincide with the location of the annular structural interconnects 82, where these particular second troughs extend all the way down to the P₂ layer 22 as well. Similarly, apertures may be patterned in the S₃ layer 24 to coincide with the location of the structural interconnects 76, where these apertures also extend all the way down to the P₂ layer 22. The P₃ layer 26 may then be deposited on top of the S₃ layer 24 to define the second plate 52, as well as into the “partially filled” annular first troughs in the S₃ layer 24 (relating to the filtering walls 60), into the annular second troughs in the S₃ layer 24 (relating to the annular structural interconnects 82), and into the apertures in the S₃ layer 24 (relating to the structural interconnects 76). The deposition of structural material into the “partially filled” annular first troughs in the S₃ layer 24 is then what defines the filtering walls 60, the deposition of structural material into the annular second troughs in the S₃ layer 24 is then what defines the annular structural interconnects 82, and the deposition of structural material into the apertures is then what defines the structural interconnects 76. The second plate 52, the filtering walls 60, the annular structural interconnects 82, and the structural interconnects 76 may then be characterized as existing in a single fabrication level (P₃ layer 26 in the noted example), since they were all defined by a deposition of a structural material before having to form any overlying layer of a sacrificial material (e.g., from a single deposition of a structural layer or film). It should be noted that at least part of the S₃ layer 24 remains between the ends of the filtering walls 60 and the first plate 44 (prior to the release).

The first plate 44 and/or the second plate 52 of the MEMS filter modules 40 could also be fabricated in multiple structural layers or films at multiple fabrication levels as noted. For instance: the first plate 44 could be fabricated in both the P₂ layer 22 and P₁ layer 18, where the P₂ layer 22 is deposited directly on at least part of the P₁ layer 18 that is to define the first plate 44 (e.g., some material of the S₂ layer 20 could be encased at one or more locations between those portions of the P₂ layer 22 and the P₁ layer 18 that are to define the first plate 44, for any appropriate purpose); the first plate 44 could be fabricated in both the P₃ layer 26 and P₂ layer 22, where the P₃ layer 26 is deposited directly on at least part of the P₂ layer 22 that is to define the first plate 44 (e.g., some material of the S₃ layer 24 could be encased at one or more locations between those portions of the P₃ layer 26 and the P₂ layer 22 that are to define the first plate 44, for any appropriate purpose); and/or the second plate 52 could be fabricated in both the P₄ layer 30 and P₃ layer 26, where the P₄ layer 30 is deposited directly on at least part of the P₃ layer 26 that is to define the second plate 52 (e.g., some material of the S₄ layer 28 could be encased at one or more locations between those portions of the P₄ layer 30 and the P₃ layer 26 that are to define the second plate 52, for any appropriate purpose). Another option would be to form a particular component of the MEMS flow module 40 in multiple structural layers or films at different fabrication levels, but that are structurally interconnected in an appropriate manner (e.g., by one or more posts, columns or the like extending between). For instance: the first plate 44 could be formed in both the P₃ layer 26 and the P₂ layer 22 with one or more structural interconnections extending therebetween (that would pass through the S₃ layer 24); the second plate 52 could be formed in both the P₄ layer 30 and the P₃ layer 26 with one or more structural interconnections extending therebetween (that would pass through the S₄ layer 28). Generally, this can be done by forming appropriate cuts or openings down through the intermediate sacrificial layer (to expose the underlying structural layer and that will define such structural interconnections once the overlying structural layer is deposited both on top of the intermediate sacrificial layer and in the noted cuts or openings therein) before depositing the overlying structural layer. In any case, the first plate 44 and second plate 52 are fabricated at different fabrication levels, but are structurally interconnected by the annular structural interconnects 82 and the structural interconnects 76.

Notwithstanding the foregoing, the various components of the MEMS flow module 40 may be formed in different layers of a MEMS structure compared to what is been described herein. Furthermore, it will be appreciated that the various complements of the MEMS flow module 40 may be formed in a reverse order to that described herein.

The general construction of one embodiment of a MEMS flow module (a MEMS device) is illustrated in FIGS. 16A-C, is identified by reference numeral 340, and provides pressure or flow regulation capabilities, filtration capabilities, or both. Although the MEMS flow module 340 is illustrated as having a circular configuration in plan view, any appropriate configuration may be utilized and in any appropriate size.

As shown in FIGS. 16A-16B, the MEMS flow module 340 includes a flow plate 350 (e.g., fabricated in P₃ layer 26) having a plurality of flow ports 352 that extend completely through the flow plate 350 and that are equally spaced about a common center point in the illustrated embodiment. Any number of flow ports 352 may be utilized, and the flow ports 352 may be of any appropriate size and/or configuration. The flow ports 352 could also be disposed in other appropriate arrangements. It would be typical to configure the MEMS flow module 340 to allow a target flow rate for a target differential pressure. The flow rate through the MEMS flow module 340 at other differential pressures would depend on the various characteristics of the MEMS flow module 340.

The MEMS flow module 340 further includes a flow controlling or regulating structure 362 (e.g., fabricated in the P₂ layer 22 or in a combined P₂ layer 22/P₁ layer 18) and an outer support ring 368 (e.g., fabricated in the P₂ layer 22 or in a combined P₂ layer 22/P₁ layer 18). That is, with the regulating structure 362 being in an undeformed state (e.g., where there is no differential pressure), the outer support ring 368 and the regulating structure 362 may be disposed in at least generally coplanar relation. The outer support ring 368 may be of any appropriate size, shape, and/or configuration. In the illustrated embodiment, the outer support ring 368 is annular in that it extends a full 360 degrees about a common point. “Annular” does not require the outer support ring 368 to be circular. The MEMS flow module 340 could include one or more additional flow plates that each have one or more flow ports. For instance, another such flow plate could be provided such that the regulating structure 362 is “sandwiched” between this additional flow plate and the flow plate 350. Any additional flow plate or flow plates could be disposed in spaced relation to another flow plate (e.g., including being fixedly interconnected therewith through one or more structural interconnections of any appropriate type) or could be disposed in interfacing relation with another flow plate (e.g., a flow plate could be fabricated in the P₄ layer 30, that in turn is deposited directly on a flow plate 350 that is fabricated in the P₃ layer 26).

The regulating structure 362 includes a center portion or support 364 and a plurality of cantilevered structures or baffles 366 that may be characterized as extending radially outwardly from the support 364 (e.g., in spoke-like fashion). It should be appreciated that the baffles 366 could extend radially inwardly from a common support as well, such as from the outer support ring 368 (not shown). That is, the support 364 provides a supporting function for the baffles 366, which cantilever from the support 364 (e.g., one end 376 of each baffle 366 is attached to the support 364, while the opposite end 378 is “free” or unsupported). Generally, both the support 364 and baffles 366 may be of any appropriate size/shape/configuration that allows each baffle 366 to flex for purposes of changing the spacing between the baffles 366 and the flow plate 350. In the illustrated embodiment, each baffle 366 flexes at least generally about an axis that is perpendicular to its length dimension (corresponding with the distance from where a particular baffle 360 attaches the support 364 and its free end 378). Removing a center portion of the support 364 (e.g., a region such as that identified by the dashed lines in FIG. 16B) of the flow regulating structure 362 may reduce the rigidity of the flow plate 350, which may be desirable for at least one or more applications. That is, removing the above-noted portion of the support 364 may allow the flow plate 350 to flex more than the configuration presented in FIG. 16B.

Any number of baffles 366 may be used, although each baffle 366 will be associated with at least one flow port 352 through the flow plate 350, and the baffles 366 may be disposed in any appropriate arrangement. In the illustrated embodiment, the baffles 366 are equally spaced about the support 364 and at least generally extend from a common location (e.g., the length dimension of each baffle 366 is disposed along a radii emanating from a common point). As shown, each baffle 366 has a free end 378 that is operable to move relative to the flow plate 350 in relation to the development of at least a certain pressure differential across the MEMS flow module 340. Further, each baffle 366 is sized to overlay (e.g., be disposed over or in overlying relation) a corresponding flow port 352 when the baffle 366 is in an adjacent relationship to the flow plate 350. Although the amount of differential pressure required to flex the baffles 366 may be of any appropriate magnitude, preferably the baffles 366 will move to at least some degree anytime the differential pressure is greater than zero or anytime there is a change in the differential pressure. Accordingly, movement of the baffles 366 relative to the flow plate 350 regulates flow through the corresponding flow ports 352. The function of the baffles 366 will be more fully discussed herein.

In the illustrated embodiment, the flow plate 350 exists in at least one fabrication level, and the regulating structure 362 exists in at least one different fabrication level (e.g., the flow plate 350 and the regulating structure 362 may be fabricated in adjacent structural layers of the MEMS device). Specifically, the flow plate 50 may be fabricated in the P₃ layer 26 and the regulating structure 62 may be fabricated in at least the P₂ layer 22 (see FIG. 11). The MEMS flow module 340 may include a ring 348 that is fixedly interconnected to the outside perimeter of the top surface of the flow plate 350 or that which is opposite the outer support ring 368. That is, an annular portion of the flow plate 350 may be “sandwiched” between the ring 348 and the outer support ring 368. This ring 348 may be a metallic ring that is attached to or formed on the flow plate 350 after the MEMS flow module 340 has been fabricated, or, may be made from another fabrication level (e.g., P₄ layer 30). Generally, the ring 348 may provide a desired interface with a housing or other structure that incorporates the MEMS flow module 340.

As will be appreciated, the various components of the MEMS flow module 340 may be formed within different layers of a MEMS structure. Furthermore, it will be appreciated that, unless otherwise stated, the various components of the MEMS flow module 340 may be formed in a MEMS structure in a reverse order as well. However, in the embodiment shown, the regulating structure 362 is formed at least in the P₂ layer 22 (also possibly in the P₁ layer 18, where the P₂ layer 22 and the P₁ layer 18 are disposed in interfacing relation) and the flow plate 350 is formed in the P₃ layer 26. Accordingly, upon the removal of the S₃ layer 24 by the release in this case, a spacing of approximately 2 microns may exist between the lower surface of the flow plate 350 and each of the upper surface of the regulating structure 362 and the upper surface of the outer support ring 368.

FIG. 16B shows an exploded, perspective view of the MEMS flow module 340. Specifically, FIG. 16B is a cross-section of the MEMS flow module 340 that is taken along a plane that is parallel to the flow plate 350, at a location that is between the flow plate 350 and the regulating structure 362 in the space between a plurality of flow-restricting rings 354 (discussed below) and the regulating structure 362, and with the flow plate 350 having been rotated or pivoted away from the regulating structure 362 and outer support ring 368. As shown, various structures are formed during the microfabrication process to interconnect the regulating structure 362 to the flow plate 350, as well as to interconnect the outer support ring 368 to the bottom perimeter of the flow plate 350. More specifically, a plurality of interconnects or anchors 370 are formed between the support 364 of the regulating structure 362 and a bottom, center portion of the flow plate 350. Any number of anchors 370 may be utilized, the anchors 370 may be of any appropriate size, shape, and configuration, and the anchors 370 may be disposed in any appropriate arrangement. Likewise, a plurality of “radially spaced” annular connectors 372 are formed between the outer support ring 368 and the bottom of the flow plate 350 at a location so as to encompass all flow ports 352. “Annular” again only means that the connectors 372 extend a full 360 degrees about a common reference point, and thereby does not limit the connectors 372 to having a circular configuration. Any number of connectors 372 may be utilized. Using multiple, radially spaced connectors 372, as shown, provides redundant radial seals, which may be desirable for one or more applications.

Consider the case where the regulating structure 362 and outer support ring 368 are fabricated at least in the P₂ layer 22 (again, typically the P₂ layer 22 and P₁ layer 18 will be disposed in interfacing relation). In this case, the anchors 370 and annular connectors 372 could be fabricated after the regulating structure 362 and outer support ring 368 have been patterned from at least the P₂ layer 22. Once these structures 362, 368 have been fabricated, the S₃ layer 24 may be deposited on top of both the regulating structure 362 and outer support ring 368, as well as into the space between the individual baffles 366 and into the space between the regulating structure 362 and the outer support ring 368. The S₃ layer 24 may then be patterned to define a plurality of holes therein that extend down to the P₂ layer 22 to correspond with the desired cross-sectional configuration and location of the anchors 370, and the S₃ layer 24 may also be patterned to define a plurality of annular trenches that extend down to the P₂ layer 22 to correspond with the desired cross-sectional configuration and location of the annular connectors 372. These holes and trenches extend all the way through the S₃ layer 24 and down to the P₂ layer 22. The P₃ layer 26 may then be deposited onto the upper surface of the S₃ layer 24 and into the holes and trenches in the S₃ layer 24. This P₃ layer 26 may then be patterned to define the perimeter of the flow plate 350 and the various flow ports 352 extending therethrough. The anchors 370, annular connectors 372, and flow plate 350 are thereby fabricated from the P₃ layer 26 and exist at a common fabrication level. Accordingly, the anchors 370 fixedly interconnect the support 364 of the regulating structure 362 to the bottom surface of the flow plate 350, and the annular connectors 372 fixedly interconnect the outer support ring 368 to a bottom of the flow plate 350.

FIG. 16C illustrates the general operation of a representative flow port 352 and a corresponding baffle 366. As shown in FIG. 16C, the flow plate 350 and baffle 366 are shown in a home or first position, or, stated another way, a pressure differential across the MEMS flow module 340 is not yet sufficient to deflect the baffle 366 away from the flow plate 350 (preferably, this is the position when there is no differential pressure across the baffles 366). In the latter regard, a first pressure PH above the flow plate 350 is not sufficiently greater than a second pressure P_{L} below the baffle 366 to result in deflection of the baffle 366 away from the flow plate 350. In this home position, the flow plate 350 and baffle 366 may be spaced approximately 2 microns apart in accordance with a typical spacing between adjacent structural/fabrication MEMS layers. While the pressure differential across the MEMS flow module 340 may not be sufficient to appreciably deflect the baffle 366, a pressure differential may still be present. Accordingly, if a 2 micron spacing were maintained between the baffle 366 and the flow plate 350, an undesired flow may proceed through the MEMS flow module 340 from the side of the first pressure P_{H} to the side of the second pressure P_{L}. Such an undesired flow may be addressed by providing an appropriate structure for each flow port 352 to create a flow restriction of a desired magnitude/amount. In the illustrated embodiment, a flow-restricting structure in the form of an annular flow-restricting wall or ring 354 is provided for each flow port 352. “Annular” means that the flow-restricting ring 354 extends a full 360 degrees about a common point, and does not limit the flow-restricting ring 354 to a circular configuration. Other types of flow-restricting structures could be utilized as well. For instance, each flow-restricting ring 354 could be replaced by a plurality of flow-restricting segments of any appropriate size/shape/configuration, where adjacent pairs of flow-restricting segments would be appropriately spaced from each other. The gap between such flow-restricting segments and the corresponding baffle 366, as well as the gap between adjacent pairs of flow-restricting segments, would provide the desired degree of flow restriction. A common flow-restricting structure could also be associated with a plurality of first flow ports 352 (e.g., a flow-restricting ring 354 or a plurality of flow restricting segments could be collectively disposed about a group of first flow ports 352).

In the case where the flow plate 350 is fabricated in a level that is further from the substrate 10 than the regulating structure 362, each annular flow-restricting ring 354 may be disposed on the bottom surface of the flow plate 350, or that surface which faces the regulating structure 362. In the case where the flow plate 350 is fabricated in a level that is closer to the substrate 10 than the regulating structure 362, each annular flow-restricting ring 354 may be disposed on the upper surface a baffle 366, or that surface which faces the flow plate 350. In either case, the function of each flow-restricting ring 354 is to reduce the size of a flow channel between the associated baffle 366 and flow port 352. In one embodiment and with the baffles 366 in an un-deflected state or in the “home” position of FIG. 16C, a gap 358 between the bottom of the flow-restricting ring 354 and its corresponding baffle 366 in the illustrated embodiment is on the order of about 0.4 microns or less. Other spacings may be appropriate, depending for instance upon the application in which the MEMS flow module 340 is being used. In one embodiment, the height of the gap 358 in the FIG. 16C configuration is no more than about 0.3 microns, although a height of about 0.1 microns or less may be desirable in at least certain instances. These same spacings may be realized/utilized when the annular flow-restricting rings 354 instead extend from the baffles 366 in the above-noted manner. Moreover, the same spacings may be realized/utilized when a particular flow-restricting ring 354 is replaced by a plurality of flow-restricting segments that are appropriately spaced from each other.

The annular flow-restricting rings 354 may be formed in conjunction with the anchors 370 and annular connectors 372. Specifically, annular troughs may be formed through the S₃ layer 24 to the P₂ layer 22 on top of each of the baffles 366. In order to separate the annular flow-restricting rings 354 from the baffles 366, a very thin layer (e.g., about 0.3 microns or less, and corresponding with desired size of the gap 358) of sacrificial material may be deposited on top of the S₃ layer 24 and at the base of these annular troughs. The thickness of this layer is definable at small dimensions. As will be appreciated, formation of the annular troughs corresponding to the annular flow-restricting rings 354 and deposition of the thin layer of sacrificial material may be performed prior to formation of the holes and annular troughs corresponding to the anchors 370 and annular connectors 372. The deposition of the thin layer of sacrificial material results, after the release, in a narrow gap 358 between the top of the baffle 366 and the bottom of the annular flow-restricting ring 354. The thickness of the deposition may be controlled such that the resulting gap 358 (between the bottom surface of the annular flow-restricting ring 354 and the top surface of the baffle 366) substantially restricts flow across the MEMS flow module 340 in the absence of the baffle 366 being deflected from the home position and away from the flow plate 350. In this regard, the size of the largest gap 358 should be no more than about 105% of the size of the smallest gap 358. Each gap 358 may also define a filter trap gap of sorts for a flow attempting to proceed between the baffles 366 and the flow plate 350. In one embodiment, each gap 358 may filter a flow through the MEMS flow module 340 when the baffles 366 are in the position illustrated in FIG. 16C, while also providing a desired flow restriction through the MEMS flow module 340. Movement of the baffles 366 away from their corresponding flow port 352 in response to the development of at least a certain differential pressure provides a pressure regulation function in that the MEMS flow module 340 will then accommodate a greater flow. When providing this pressure regulation function, the flow-restricting rings 354 may not be providing any significant filtering function. For at least certain applications, the primary function of the flow-restricting rings 354 is to limit the flow rate through the MEMS flow module 340, and not provide a filtering function. Again, however, the flow-restricting rings 354 may provide a filtering function as desired/required.

The gap 358 may be designed such that the annular flow-restricting ring 354 and its corresponding baffle 366 are spaced to allow at least a certain flow through the MEMS flow module 340 without requiring any deflection of the baffles 366. That is, the MEMS flow module 340 may be designed to provide a constantly open flow path that allows at least a certain limited flow through the MEMS flow module 340 at all times. Such a constantly open flow path may be beneficial in at least number of respects. One relates to the case where the MEMS flow module 340 is used to relieve intraocular pressure in an eye (e.g., by being incorporated into an eye implant). In this case, the flow plate 350 of the MEMS flow module 340 could be on the “anterior chamber” side (e.g., the flow of aqueous humor out of the anterior chamber of the patient's eye through the MEMS flow module 340 would be through one or more flow ports 352, and then through the spacing between the baffles 366 and the flow plate 350, and then ultimately out of the MEMS flow module 340). Having the open flow path exist at all times (such that it always has a volume greater than zero) is believed to at least generally mimic the flow of aqueous humor out of the anterior chamber of a patient's eye through the eye's canal of Schlemm. However, the MEMS flow module 340 could be designed so that the baffles 366 are actually disposed directly on their corresponding annular flow-restricting ring 354 until at least a certain differential pressure exists (e.g., a differential pressure “set point”, which may in fact be zero as noted), after which the baffles 366 then would move into spaced relation with the corresponding annular flow-restricting ring 354 to open the flow path.

Each baffle 366 is interconnected at its base or fixed end 376 to the support 364 of the regulating structure 362. See FIGS. 16B and 16C. Opposite of the fixed base 376 is a free end 378 of the baffle 366. The free end 378 of the baffle 366 is operative to move along an at least generally arcuate path in response to the baffle 366 experiencing at least a certain differential pressure. More specifically, the baffle 366 flexes in response to at least a certain pressure differential that exists across the MEMS flow module 340. If the pressure acting on the side of a particular baffle 366 that faces its corresponding flow port 352 is greater than the pressure acting on the opposite side of this baffle 366 by at least a certain amount, this pressure differential will result in a force that is applied to the baffle 366 that is operative to flex the baffle 366 downward in the view shown in FIG. 16C. That is, the baffle 366 flexes away from its corresponding flow port 352 and annular flow-restricting ring 354 to further open a flow path segment within the MEMS flow module 340. This flexing also stores forces or creates stresses in each baffle 366 that may be used to return the same either back toward or to the position illustrated in FIG. 16C as the magnitude of the pressure differential is reduced. That is, the baffles 366 preferably elastically deform as the pressure differential increases above a certain amount, and the elasticity of the baffles 366 may provide a restoring force that at least contributes to the movement of the baffles 366 back toward or to their respective home position (e.g., FIG. 16C), depending upon the magnitude of the reduction of the pressure differential.

The volume of a flow path segment is at least partially dependent upon the flexure of the baffle 366. The further the baffle 366 is flexed away from its corresponding flow port 352, the greater the volume of the flow path segment will be (e.g., up to a certain maximum). Importantly, the movement of the baffle 366 allows the flow rate through the flow port 352 to increase greater than proportionally to an increase in the pressure differential across the MEMS flow module 340. The maximum distance that the baffle 366 is allowed to move away from the flow plate 350 may be controlled, such as by using an appropriate travel limiter or the like (e.g., a mechanical “catch”).

Typically the MEMS flow module 340 will be used in an application where a high pressure source P_{H} (e.g., the anterior chamber of a patient's eye) acts on the top of the flow plate 350 or that surface of the flow plate 350 which projects or faces away from the regulating structure 362, while a typically lower pressure source P_{L} (e.g., the environment) acts on the bottom of the flow plate 350 or that surface of the flow plate 350 which projects toward or faces the regulating structure 362. A change in the pressure from the high pressure source P_{H} may cause one or more of the baffles 366 to move further away from the flow plate 350, which thereby increases the flow rate through the MEMS flow module 340. Preferably, a very small change in the pressure from the high-pressure source P_{H} will allow for greater than a linear change in the flow rate out of the MEMS flow module 340 through the flow ports 352 and past the baffles 366. For instance, a small increase in the pressure of the high pressure source P_{H} may increase the deflection of the baffles 366 (i.e., such that they move further away from the annular flow-restricting rings 354) to provide more than a linear increase in the flow rate through the MEMS flow module 340. That is, there is preferably a non-linear relationship between the flow rate passing through the MEMS flow module 340 and a change in the differential pressure being experienced by the MEMS flow module 340. The flow rate through the flow path segment defined by the space between the baffles 366 and the annular flow-restricting rings 354 should be a function of the cube of the height of this flow path segment, or the gap 358 between the baffles 366 and their corresponding annular flow-restricting ring 354 (at least in the case of laminar flow, which is typically encountered at these dimensions and flow rates). Stated another way, the development of at least a certain change in the differential pressure across a particular baffle 66 will provide greater than a linear increase in the volume of the flow channel segment between the flow-restricting ring 354 and its corresponding baffle 366.

Consider the case where the MEMS flow module 340 is used in an implant to regulate the pressure in the anterior chamber of a patient's eye that is diseased, and where it is desired to maintain the pressure within the anterior chamber of this eye at about 5 mm of HG. The stiffness of the baffles 366 may be configured such that they will adjust the flow rate out of the anterior chamber and through the MEMS flow module 340 such that the maximum pressure within the anterior chamber of the patient's eye should be no more than about 7-8 mm of HG (throughout the range for which the MEMS flow module 340 is designed). Stated another way, the stiffness of the baffles 366 allows for maintaining at least a substantially constant pressure in the anterior chamber of the patient's eye (the high pressure source P_{H} in this instance), at least for a reasonably anticipated range of pressures within the anterior chamber of the patient's eye.

In order to regulate the pressure differential across and/or flow through the MEMS flow module 340, one or more characteristics of the flow ports 352 and/or baffles 366 may be adjusted. As will be appreciated, the force applied to each baffle 366 by a differential pressure is proportional to the area of the corresponding flow port 352. Accordingly, by adjusting the size (e.g., diameter) of the flow ports 352, the force applied to the baffles 366 for a given pressure differential may be increased and/or decreased. Likewise, the stiffness of the baffles 366 may be designed for a particular application. In this regard, the baffles 366 can be likened to a beam having a fixed base 376 and a free end 378. By adjusting the width, height, cross-sectional shape and/or length of such a beam, the stiffness the baffle 366 may be adjusted. The stiffness of the baffles 366 will of course have an effect on the magnitude of the differential pressure that must exist to start flexing the baffles 366.

There are a number of features and/or relationships that contribute to the pressure or flow regulation function of the MEMS flow module 340, and that warrant a summarization. First is that the MEMS flow module 340 is an autonomous or self-contained device. No external power is required for operation of the MEMS flow module 340. Stated another way, the MEMS flow module 340 is a passive device—no external electrical signal of any type need be used to move the baffles 366 relative to the flow plate 350 for the MEMS flow module 340 to provide its pressure or flow regulation function. Instead, the position of the baffles 366 relative to the flow plate 350 is dependent upon the differential pressure being experienced by the baffles 366, and the flow rate out of the MEMS flow module 340 (through the space between adjacent baffles 366 and/or the space between the baffles 366 and the outer support ring 368) is in turn dependent upon the position of the baffles 366 relative to the flow plate 350 (the spacing therebetween (e.g., gap 358), and thereby the size of this flow path segment). Finally, it should be noted that the MEMS flow module 340 may be designed for a laminar flow therethrough, although the MEMS flow module 340 may also be applicable for a turbulent flow therethrough as well.

As will be appreciated, prior to the release of the MEMS flow module 340, at least one sacrificial layer (e.g., the S₃ layer 24) will be disposed between the flow plate 350 and the regulating structure 362, while at least one sacrificial layer (e.g., the S₁ layer 16) will be disposed on the side of the regulating structure 362 that is opposite that which faces the flow plate 350. In order to remove these sacrificial layers, a plurality of etch release holes may be formed through the flow plate 350 and through the regulating structure 362 in order to reduce the amount of time required to remove these sacrificial layers. Typically these etch release holes will have a diameter of no more than about one micron. At least certain lithographic techniques only permit the formation of an etch release hole having a diameter on the order of about one micron. As will be appreciated, such etch release holes will remain in the resulting MEMS flow module 340. There are a number of potential disadvantages associated with etch release holes of this size for the MEMS flow module 340. One is that the existence of a number of etch release holes of this size may provide an undesirably high minimum flow rate through the MEMS flow module 340. That is, etch release holes of this size could possibly have an undesired effect on the flow or pressure regulating capabilities of the MEMS flow module 340. Another is that potentially undesirable contaminants having a size of about one micron or less may pass through the MEMS flow module 340 by passing through such etch release holes.

In cases where the diameter of the etch release holes cannot be made sufficiently small (e.g., a diameter of no more than about 0.2 or 0.3 microns), and possibly depending upon the location of a particular etch release hole in the MEMS flow module 340, a flow-restricting structure or a flow restrictor may be provided in relation to one or more of these etch release holes. A single flow restrictor may be associated with a single etch release hole in a given fabrication level, or may be associated with multiple etch release holes in a given fabrication level. In the case of the MEMS flow module 340, a flow restrictor may be provided for each etch release hole through the flow plate 350. However, a flow restrictor may only be required for those etch release holes through the baffles 366 that are aligned with or encompassed by a corresponding flow port 352 in the flow plate 350. A flow restrictor could be provided for each etch release hole utilized by the MEMS flow module 340, or for any number of etch release holes utilized by the MEMS flow module 340. For instance, a flow restrictor may be used for a certain percentage of the etch release holes through the flow plate 350, and again possibly only for those etch release holes through the baffles 366 that are aligned with or encompassed by a corresponding flow port 352 in the flow plate 350. However, a flow restrictor could be used in relation to any number of etch release holes through a particular baffle 366.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

What is claimed is:

1. A method for addressing intraocular pressure within an eye, comprising the step of:
providing a drainage flow path from an anterior chamber of an eye to a first drainage location, wherein said drainage flow path allows a flow of at least about 0.15 microliters/minute/mm²/mm-Hg through said drainage flow path.

2. The method of claim 1, wherein said first drainage location is exteriorly of the eye.

3. The method of claim 1, wherein said providing step comprises directing a first conduit section of a conduit through said eye and into said anterior chamber, wherein an entirety of a second conduit section of said conduit is disposed exteriorly of said eye, and wherein said first conduit section comprises a first cross-sectional profile taken perpendicularly to a length dimension of said first conduit section that is smaller than a second cross-sectional profile of said second conduit section taken perpendicularly to a length dimension of said second conduit section.

4. The method of claim 3, wherein a flow module is disposed within said second conduit section.

5. The method of claim 4, wherein said flow module is selected from the group consisting of a filter, a pressure regulator, and a device comprising filter and pressure regulator sections.

6. The method of claim 4, further comprising step of replacing said flow module, wherein said replacing step comprises expanding at least a portion of said second conduit section.

7. The method of claim 3, wherein first and second flow modules are disposed within said second conduit section.

8. The method of claim 7, wherein said first flow module is a filter and wherein said second flow module is a pressure regulator.

9. The method of claim 7, wherein said first flow module allows an at least substantially linear increase in a flow through said first flow module in response to an increase in a differential pressure across said first flow module, and wherein said second flow module allows greater than a linear increase in a flow through said second flow module in response to an increase in a differential pressure across said second flow module.

10. The method of claim 7, wherein said first and second flow modules are disposed in series within said second conduit section.

11. The method of claim 3, wherein said second conduit section is entirely disposed within a conjunctival cul-de-sac.

12. The method of claim 3, wherein said first and second conduit sections are integrally formed.

13. The method of claim 12, wherein a flow module is disposed within said second conduit section.

14. The method of claim 13, further comprising step of replacing said flow module, wherein said replacing step comprises expanding at least a portion of said second conduit section.

15. The method of claim 12, wherein a third conduit section is interconnected with said second conduit section by a coupling, and wherein a flow module is disposed within said third conduit section.

16. The method of claim 15, wherein an outer wall of said coupling comprises first and second protuberances, wherein said second conduit section is disposed over said first protuberance and wherein said third conduit section is disposed over said second protuberance.

17. The method of claim 15, further comprising the step of replacing said flow module, wherein said replacing step comprises disconnecting at least one of said coupling from said second conduit section and disconnecting said third conduit section from said coupling, wherein said flow module remains within said third conduit section during said replacing step.

18. The method of claim 3, wherein a third conduit section is interconnected with said second conduit section by a coupling, and wherein a flow module is disposed within said third conduit section.

19. The method of claim 18, further comprising the step of replacing said flow module, wherein said replacing step comprises disconnecting at least one of said coupling from said second conduit section and disconnecting said third conduit section from said coupling, wherein said flow module remains within said third conduit section during said replacing step.

20. The method of claim 3, wherein said first and second conduit sections each comprise a cylindrical outer wall.

21. The method of claim 1, wherein said providing step comprises directing a conduit through said eye and into said anterior chamber.

22. The method of claim 21, further comprising the step of applying an anti-bacterial material to said eye before said directing step.

23. The method of claim 21, wherein at least a portion of an exterior of said conduit promotes adhesion with adjoining biological tissue.

24. The method of claim 23, wherein said biological tissue is selected from the group consisting of the conjunctiva and the sclera of said eye.

25. The method of claim 21, wherein a first end of said conduit is disposed within said anterior chamber and a second end of said conduit is disposed within a conjunctival cul-de-sac.

26. The method of claim 21, wherein a flow module is disposed within said conduit.

27. The method of claim 26, wherein said flow module is selected from the group consisting of a filter, a pressure regulator, and a device comprising filter and pressure regulator sections.

28. The method of claim 26, wherein said flow module comprises at least one hydrophilic surface.

29. The method of claim 26, wherein said flow module comprises means for reducing the ability of biological material to attach to said flow module.

30. The method of claim 26, wherein at least one surface of said flow module that is exposed to a fluid within said drainage flow path comprises a self-assembled monolayer coating.

31. The method of claim 26, wherein said flow module accommodates greater than a linear increase in a flow through said flow module in response to an increase in a differential pressure across said flow module.

32. The method of claim 26, wherein said flow module accommodates at least a substantially linear increase in a flow through said flow module in response to an increase in a differential pressure across said flow module.

33. The method of claim 26, wherein each flow path through said flow module is of an at least substantially fixed dimension.

34. The method claim 26, wherein said flow module comprises at least one element that moves in response to a change in a differential pressure to which said at least one element is exposed.

35. The method of claim 26, further comprising the step of at least substantially occluding said conduit and thereafter replacing said flow module.

36. The method of claim 35, further comprising the steps of applying an anti-bacterial material to said eye before said replacing step.

37. The method of claim 35, wherein a first portion of said conduit is disposed within said eye and a second portion of said conduit is disposed exteriorly of said eye, wherein said flow module is disposed within said second portion of said conduit, and wherein said replacing step is executed without removing said first portion of said conduit from said eye.

38. The method of claim 26, wherein a first portion of said conduit is disposed within said eye and a second portion of said conduit is disposed exteriorly of said eye, wherein said flow module is disposed within said second portion of said conduit, and wherein said method further comprises the step of replacing said flow module without removing said first portion of said conduit from said eye.

39. The method of claim 38, further comprising the step of at least substantially occluding said conduit before said replacing step.

40. The method of claim 38, further comprising the step of applying an anti-bacterial material to said eye before said replacing step.

41. The method of claim 21, wherein first and second flow modules are disposed within said conduit.

42. The method of claim 41, wherein said first flow module is a filter in wherein said second flow module is a pressure regulator.

43. The method of claim 41, wherein said first flow module accommodates at least a substantially linear increase in a flow through said first flow module in response to an increase in a differential pressure across said first flow module, and wherein said second flow module accommodates greater than a linear increase in a flow through said second flow module in response to an increase in a differential pressure across said second flow module.

44. The method of claim 41, wherein said first and second flow modules are disposed in series within said conduit.

45. The method of claim 1, wherein said drainage flow path allows a flow of at least about 0.30 microliters/minute/mm²/mm-Hg through said drainage flow path.

46. The method of claim 1, wherein said drainage flow path allows a flow of at least about 0.6 microliters/minute/mm²/mm-Hg through said drainage flow path.

47. The method of claim 1, wherein said drainage flow path allows a flow of at least about 1.2 microliters/minute/mm²/mm-Hg through said drainage flow path.

48. The method of claim 1, wherein said drainage flow path allows a flow of about 1.5 microliters/minute/mm²/mm-Hg through said drainage flow path.

49. The method of claim 1, wherein said drainage flow path comprises a first region, wherein said first region is located outside of said anterior chamber of said eye, and wherein said first region is configured to retain particles having a minimum dimension of at least about 0.4 microns.

50. The method of claim 1, wherein said drainage flow path comprises a first region, wherein said first region is located outside of said anterior chamber of said eye, and wherein said first region is configured to retain particles having a minimum dimension of at least about 0.3 microns.

51. The method of claim 1, wherein said drainage flow path comprises a first region, wherein said first region is located outside of said anterior chamber of said eye, and wherein said first region is configured to retain particles having a minimum dimension of at least about 0.2 microns.

52. The method of claim 1, wherein said drainage flow path comprises a first region, wherein said first region is located outside of said anterior chamber of said eye, and wherein said first region is configured to retain particles having a minimum dimension of at least about 0.1 microns.

53. The method of claim 1, wherein said drainage flow path comprises a first region, wherein said first region is configured to retain pseudomonas aeruginosa.

54. The method of claim 1, wherein said drainage flow path comprises a first region, wherein said first region is configured to retain staphylococcus aureus.

55. The method of claim 1, wherein said drainage flow path comprises a first region, wherein said first region is configured to retain brevundimonas diminuta.

56. A method for addressing intraocular pressure within an eye, comprising the steps of:
occluding a portion of a conduit that extends from an anterior chamber of an eye to a first drainage location; and
replacing a flow module disposed within said conduit after said occluding step.

57. A system for addressing intraocular pressure within an eye, comprising:
a conduit comprising a drainage flow path, wherein a first end of said conduit is directed through the eye to fluidly interface with the anterior chamber of the eye when said system is installed, and wherein a second end of said conduit is disposed outside of the anterior chamber when said system is installed; and
means for allowing a flow of at least about 0.15 microliters/minute/mm²/mm-Hg out of said anterior chamber through said drainage flow path.

58. A system for addressing intraocular pressure within an eye, comprising:
a conduit comprising a drainage flow path;
a first flow module disposed within said drainage flow path; and
a second flow module disposed within said drainage flow path.

59. A system for addressing intraocular pressure within an eye, comprising:
an integral first conduit comprising a drainage flow path, wherein said first conduit comprises first and second conduit sections, wherein said first conduit section comprises a first cross-sectional profile taken perpendicularly to a length dimension of said first conduit section that is smaller than a second cross-sectional profile of said second conduit section taken perpendicularly to a length dimension of said second conduit section, wherein an end of said first conduit section is directed through the eye when said system is installed, and wherein an entirety of said second conduit is disposed exteriorly of the eye when said system is installed.

60. A system for addressing intraocular pressure within an eye, comprising:
a conduit comprising a drainage flow path, wherein a first end of said conduit is directed through the eye to fluidly interface with the anterior chamber of the eye when said system is installed, and wherein a second end of said conduit is disposed outside of the anterior chamber when said system is installed;
a MEMS flow module disposed within said conduit, wherein said MEMS flow module comprises a plurality of gaps through which a flow may progress, wherein a size of a largest of said plurality of gaps is no more than about 105% of a size of a smallest of said plurality of gaps.

61. A method for reducing intraocular pressure in an eye, said method comprising providing a drainage path from an anterior chamber to an external location, wherein the drainage path allows the outflow of aqueous fluid at a rate of at least 1.5 microliters/minute in response to an ocular pressure of 10 mm-Hg or higher while preventing the intrusion of substantially all bacteria having a dimension of 0.35 micrometer or larger.

62. The method of claim 61, wherein the drainage path has a cross-sectional area no greater than 1 mm².

63. The method of claim 61, wherein a filter is disposed within the drainage path.

64. A method for treating glaucoma, said method comprising draining aqueous fluid at a rate of at least 1.5 microliters/minute whenever an intraocular pressure exceeds 10 mm-Hg while substantially completely excluding the intrusion of Pseudomonas aeruginosa and larger bacteria.

65. A system for reducing intraocular pressure in an eye, said system comprising:
a conduit structure adapted to be implanted from an anterior chamber of an eye to a location external of the eye; and
a filter structure in the conduit structure, said filter structure allowing the outflow of aqueous fluid at a rate of at least 1.5 microliters/minute in response to an ocular pressure of 10 mm-Hg or higher while preventing the intrusion of substantially all bacteria having a dimension of 0.35 micrometer or larger.

66. The system of claim 65, wherein a flow path through the conduit structure has a cross-sectional area no greater than 1 mm².
